(19) United States
(12) Patent Application Publication
(10) Pub. No.: US 2014/0142404 A1
(43) Pub. Date: May 22, 2014

(54) SINGLE-CELL LABEL-FREE PHOTOACOUSTIC FLOWOXIGRAPHY IN VIVO

(71) Applicant: The Washington University, St. Louis, MO (US)

(72) Inventors: Lihong V. Wang, Creve Coeur, MO (US); Konstantin Maslov, St. Louis, MO (US); Lidai Wang, St. Louis, MO (US)

(73) Assignee: The Washington University, St. Louis, MO (US)

(21) Appl. No.: 14/164,117

(22) Filed: Jan. 24, 2014

Related U.S. Application Data
(63) Continuation-in-part of application No. 13/125,522, filed on Apr. 21, 2011.
(60) Provisional application No. 61/107,845, filed on Oct. 23, 2008.
(60) Provisional application No. 61/756,092, filed on Jan. 24, 2013.

(51) Int. Cl. A61B 8/00; A61B 5/145; A61B 5/00

(52) U.S. Cl. CPC A61B 8/4416; A61B 5/0062; A61B 5/14542
USPC 600/324; 600/476; 600/437; 600/445

(57) ABSTRACT

A single-RBC photoacoustic flowoxigraphy (FOG) device is described that delivers laser pulses of two different wavelengths separated by a pulse separation period of about 20 μs. This separation period is sufficiently brief to enable pulses of two different wavelengths to illuminate the same single moving RBC. The acoustic signals elicited by the single RBC in response to the laser pulses of two different wavelengths may be analyzed using pulse oximetry methods similar to those described herein above to simultaneously determine a variety of functional parameters.

### CROSS-REFERENCE TO RELATED APPLICATIONS

This application is a continuation-in-part of U.S. Non-Provisional application Ser. No. 13/125,522 filed on Apr. 21, 2011 and entitled “Reflection-Mode Photoacoustic Tomography Using a Flexibly-Supported Cantilever Beam”, which is a national stage entry of PCT Application No. PCT/US09/61435 filed on Oct. 21, 2009 and entitled “Reflection-Mode Photoacoustic Tomography Using a Flexibly-Supported Cantilever Beam”, which claims priority to U.S. Provisional Application No. 61/107,845 filed on Oct. 23, 2008 and entitled “Reflection-Mode Photoacoustic Tomography Using a Flexibly-Supported Cantilever Beam”, of which all disclosures are hereby incorporated by reference in their entirety. This application further claims priority to U.S. Provisional Application No. 61/756,092 filed on Jan. 24, 2013 and entitled “Single-cell label-free photoacoustic flowoxigraphy in vivo”, the disclosure of which is also hereby incorporated by reference in its entirety.

### GOVERNMENTAL SUPPORT

This invention was made with government support under Grant No. R01 EB000712 awarded by the U.S. National Institutes of Health. The government has certain rights in the invention.

### FIELD OF THE INVENTION

The subject matter disclosed herein relates generally to photoacoustic imaging and, more specifically, to using photoacoustic tomography to characterize a target or targeted area within a tissue.

### BACKGROUND OF THE INVENTION

Most living cells require oxygen to metabolize nutrients into usable energy. In vivo imaging of oxygen transport and consumption at high spatial and temporal resolution is required to understand the metabolism of cells and related functionalities. Although individual parameters such as sO₂, partial oxygen pressure (pO₂), or blood flow speed (V_{flow}) may partially characterize tissue oxygenation, no single parameter can provide a comprehensive view of oxygen transport and consumption. To quantify the fundamental metabolic rate of oxygen (MRO₂), three primary imaging modalities have been employed in previous research: positron emission tomography (PET), functional magnetic resonance imaging (fMRI), and diffuse optical tomography (DOT). These three imaging modalities are capable of imaging MRO₂ at a millimeter-scale spatial resolution, but this resolution is inadequate to visualize MRO₂ at a single-cell resolution, at which many important oxygen transport and delivery processes occur.

Photoacoustic (PA) microscopy has been proposed to measure MRO₂ of a region at the feeding and draining blood vessels. However, this assessment of MRO₂ has been limited to a relatively large region due to the limitations of existing PA microscopy devices. As a result, the feeding and draining blood vessels—especially those surrounding a tumor—may be numerous and difficult to identify. Since micrometer-sized RBCs are the fundamental elements for delivering most of the oxygen to cells and tissues, there exists a need for direct functional imaging of flowing individual RBCs in real time.

### BRIEF SUMMARY OF THE INVENTION

In one aspect, a device for real-time spectral imaging of single moving red blood cells in a subject in vivo is provided. The device includes: an isosbestic laser to deliver a series of isosbestic laser pulses at an isosbestic wavelength, an isosbestic pulse width of less than about 10 ns and an isosbestic pulse repetition rate of at least 2 kHz; a non-isosbestic laser to deliver a series of non-isosbestic laser pulses at a non-isosbestic wavelength, a non-isosbestic pulse width of less than about 10 ns and a non-isosbestic pulse repetition rate of at least 2 kHz; an optical fiber to direct the series of isosbestic laser pulses and the series of non-isosbestic laser pulses to an optical assembly; an optical assembly to focus the series of isosbestic laser pulses and the series of series of non-isosbestic laser pulses into a beam with a beam cross-sectional diameter of less than about 10 μm through an optical focus region; and a laser controller to trigger the delivery of each isosbestic laser pulse and each non-isosbestic laser pulse. Each isosbestic laser pulse is delivered at a pulse separation period of about 20 μs before or after each adjacent non-isosbestic laser pulse. The isosbestic wavelength may be a wavelength with a hemoglobin absorbance that is essentially equal to an oxyhemoglobin absorbance and may include 532 nm, 548 nm, 568 nm, 587 nm, and 805 nm. The non-isosbestic wavelength may be any wavelength with the hemoglobin absorbance that is not equal to the oxyhemoglobin absorbance. The isosbestic wavelength may be about 532 nm and the non-isosbestic wavelength may be about 560 nm. The optical assembly may include a pair of optical lenses that may be two achromatic doublets with a numerical aperture in water of about 0.1. The device may further include a focused ultrasound transducer with an acoustic focus region that is aligned with the optical focus region and a central frequency of at least 10 MHz. The central frequency may be about 50 MHz and the focused ultrasound transducer may have an axial spatial resolution of about 15 μm. The device may further include a linear scanner to move the optical assembly and the focused ultrasound transducer in a linear scanning pattern. The linear scanner may be a voice-coil scanner with a scanning rate of at least 100 linear scans per second. The device may further include an acoustically transparent optical reflector to transmit acoustic signals from the acoustic focus region to the focused ultrasound transducer and to reflect the series of isosbestic and non-isosbestic laser pulses from the optical assembly to the optical focus region. The acoustically transparent optical reflector may include a first prism and a second prism. A first face of the first prism and a second face of the second prism may be arranged on opposite sides of an aluminum layer forming a planar optical reflector aligned at an angle of 45° relative to an axis of the optical assembly.

In another aspect, a system for real-time spectral imaging of single moving red blood cells in a subject in vivo is provided. The system includes: a dual wavelength light source module to produce a series of isosbestic laser pulses at an isosbestic wavelength, an isosbestic pulse width of less than about 10 ns and an isosbestic pulse repetition rate of at least 2 kHz and a series of non-isosbestic laser pulses at a non-isosbestic wavelength, a non-isosbestic pulse width of less than about 10 ns and a non-isosbestic pulse repetition rate of at least 2 kHz; an optical module to direct the series of isosbestic laser pulses and the series of non-isosbestic laser pulses through an optical focus region in a cylindrical beam with a beam cross-sectional diameter of less than about 10 μm; and a laser control module to trigger the delivery of each isosbestic laser pulse and each non-isosbestic laser pulse, wherein each isosbestic laser pulse is delivered at a pulse separation period of about 20 μs before or after each adjacent non-isosbestic laser pulse. The dual wavelength light source module may include an isosbestic laser to produce the series of isosbestic laser pulses and a non-isosbestic laser to produce the series of non-isosbestic laser pulses. The wavelength may be a wavelength with a hemoglobin absorbance that is essentially equal to an oxyhemoglobin absorbance and the isosbestic wavelength may be chosen from 532 nm, 548 nm, 568 nm, 587 nm, and 805 nm. The non-isosbestic wavelength may be any wavelength with the hemoglobin absorbance that is not equal to the oxyhemoglobin absorbance. The isosbestic wavelength may be about 532 nm and the non-isosbestic wavelength may be about 560 nm. The optical module may include an optical fiber operatively connected to the isosbestic laser and the non-isosbestic laser at a first end and operatively connected to a pair of optical lenses comprising two achromatic doublets with a numerical aperture in water of about 0.1 at a second end opposite to the first end of the optical fiber. The system may also include an ultrasound detection module to detect acoustic signals generated within the optical focus region in response to the series of isosbestic and non-isosbestic laser pulses. The ultrasound detection module may include a focused ultrasound transducer with a central frequency of about 50 MHz and an ultrasound focus region that is aligned with the optical focus region. The optical module may also include an acoustically transparent optical reflector to transmit acoustic signals from the acoustic focus region to the focused ultrasound transducer and to reflect the series of isosbestic and non-isosbestic laser pulses from the optical assembly to the optical focus region. The system may also include a scanning module to move the optical module and the ultrasound detection module in a linear scanning pattern. The scanning module may include a voice-coil scanner with a scanning rate of at least 100 linear scans per second. The system may obtains images of the single moving red blood cells at an axial spatial resolution of about 15 μm and a lateral spatial resolution of about 3.4 μm. The system may simultaneously obtain one or more functional parameters of the single moving red blood cells using a pulse oximetry method. The one or more functional parameters may include: total hemoglobin concentration, oxygen saturation, gradient of oxygen saturation, flow speed, metabolic rate of oxygen, and any combination thereof.

### BRIEF DESCRIPTION OF THE DRAWINGS

The following drawings illustrate various aspects of the disclosure.

FIG. 1 is a block diagram of an imaging system that includes an ultrasonic imaging system and a photoacoustic scanner.

FIG. 2 is a schematic diagram of an exemplary direct contact dark-field photoacoustic microscopy scanner.

FIG. 3 is a block diagram of an exemplary quantitative spectroscopic measurement system that includes the photoacoustic microscopy scanner shown in FIGS. 1 and 2.

FIG. 4 is a timing diagram for photoacoustic imaging used by the scanner shown in FIGS. 1-3.

FIG. 5 is a schematic diagram of an exemplary photoacoustic head that may be used with the measurement system shown in FIG. 3, including a single-element spherically focusing transducer.

FIG. 6 is a schematic diagram of a second exemplary photoacoustic head that may be used with the measurement system shown in FIG. 3, including a spherically focusing annular transducer array.

FIG. 7 is a schematic diagram of a third exemplary photoacoustic head that may be used with the measurement system shown in FIG. 3, including a linear phase array of ultrasonic transducers.

FIG. 8 is a schematic diagram of an exemplary photoacoustic scanner system that uses cantilever beam-based two-dimensional scanning for volumetric imaging.

FIG. 9 is a schematic diagram of a second exemplary photoacoustic scanner system that combines cantilever beam scanning and linear translation scanning for volumetric imaging.

FIG. 10A shows a blood flow image in a mouse prostate taken by an ultrasonic system.

FIG. 10B shows a blood oxygenation level image acquired with photoacoustic imaging.

FIG. 11A shows an ultrasonic image of blood vessels.

FIG. 11B shows a photoacoustic image of oxygen saturation of hemoglobin (SO₂).

FIG. 110 shows an ex-vivo microsphere-perfusion image of arterioles (red) and venules (blue).

FIG. 12 is a flowchart illustrating an exemplary photoacoustic tomography imaging method.

FIG. 13 is a flowchart illustrating an exemplary method for determining an oxygen metabolic rate within a biological tissue.

FIG. 14 is a schematic diagram of a single red blood cell (RBC) photoacoustic flowoxigraphy (FOG) device.

FIG. 15A is a series of images of single RBCs releasing oxygen in a capillary in a mouse brain obtained using a single red blood cell (RBC) photoacoustic flowoxigraphy (FOG) device; scale bars: x=10 μm, z=30 μm. FIG. 15B includes a series of graphs summarizing simultaneous measurements of multiple functional parameters from the images of single RBCs, including total hemoglobin concentration (C_{Hb}), oxygen saturation (sO₂), flow speed (V_{flow}, unit: mm·s⁻¹), and metabolic rate of oxygen (MRO₂). FIG. 15C is a graph summarizing normalized MRO₂ versus ∇sO₂ at various flow speeds within a vessel. FIG. 15D is a graph summarizing normalized MRO₂ versus <sO₂> at various flow speeds within a vessel; <sO₂> denotes the sO₂ averaged over the capillary segment in the field of view. FIG. 15E is a graph summarizing <sO₂> versus V_{flow} as a function of ∇sO₂. In FIGS. 15C-15E, each point on the graphs represents one measurement averaged over 1 s.

FIG. 16A is a series of images summarizing the dynamic imaging of single-RBC oxygen delivery under a transition from hypoxia to hyperoxia for 60 s as RBCs flow in the positive x-direction through a 30-μm capillary segment obtained using a single red blood cell (RBC) photoacoustic flowoxigraphy (FOG) device; each oblique line in the x-t images tracks one single RBC. FIGS. 16B-16F are graphs summarizing <C_{Hb}>, <sO₂>, ∇sO₂, V_{flow}, and MRO₂ averaged over 10 sec, respectively; error bars are SEM, P values were determined by two-way ANOVA tests, and *** indicates p<0.001.

FIG. 17A is a sO₂ maximum-amplitude-projection (MAP) image of a mouse brain cortex obtained using a single red blood cell (RBC) photoacoustic flowoxigraphy (FOG) device; the dashed box within the image encloses a capillary segment of interest and the scale bar equals 200 μm. FIG. 17B is a graph summarizing the measured systemic blood glucose level measured every 10 minutes after insulin injection. FIGS. 17C-17G are graphs summarizing MRO₂, <C_{Hb}>, <sO₂>, ∇sO₂, and V_{flow} quantified from single-RBC images of the capillary segment of interest. FIG. 17H is a graph comparing average MRO₂, <C_{Hb}>, <sO₂>, ∇sO₂, and V_{flow} during normoglycemia and hypoglycemia; error bars are SEM. FIG. 17I is a graph comparing the fitted slopes of MRO₂, <C_{Hb}>, <sO₂>, ∇sO₂, and V_{flow} during normoglycemia and hypoglycemia; error bars are SD.

FIG. 18A is a schematic of an experimental setup used for imaging of neuron—single-RBC coupling in mouse visual cortex using a single red blood cell (RBC) photoacoustic flowoxigraphy (FOG) device. FIG. 17B are images summarizing the transient responses of sO₂, ∇sO₂, V_{flow}, and MRO₂ to a single visual stimulation; error bars denote SEMs, and *: p<0.05, **: p<0.01, ***: p<0.001 according to two-way ANOVA tests. FIG. 18C is a MAP image of sO₂ obtained without continuous visual stimulations. FIG. 18D is a MAP image of sO₂ obtained with 1 Hz continuous optical flashing stimulations on the left mouse eye. The scale bars in FIGS. 18C and 18D are x=10 μm and t=10 s. FIG. 18E is a graph summarizing the relative changes of single RBC functional parameters (<C_{Hb}>, <sO₂>, ∇sO₂, V_{flow}, and MRO₂) under continuous visual stimulation; all values are normalized to mean values of control images, error bars are SEMs, and *: p<0.05 according to two-way ANOVA tests. FIGS. 18F-18I are graphs summarizing cumulative distribution functions (CDFs) of <sO₂>, ∇sO₂, V_{flow}, and MRO₂, respectively under control (ctrl) and stimulation (stim) conditions; MRO₂ was normalized to the mean value of the control experiment.

FIG. 19 is a block diagram illustrating the arrangement of modules of a single red blood cell (RBC) photoacoustic flowoxigraphy (FOG) system.

FIG. 20 is an illustration of an acoustically transparent optical reflector in an aspect.

Corresponding reference characters and labels indicate corresponding elements among the views of the drawings. The headings used in the figures should not be interpreted to limit the scope of the claims.

### DETAILED DESCRIPTION

While the making and using of various embodiments of the invention are discussed in detail below, it should be appreciated that the embodiments of the invention provides many applicable inventive concepts that may be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the embodiments of the invention. Terms such as “a,” “an” and “the” arc not intended to refer to only a singular entity, but include the general class of which a specific example may be used for illustration. The terminology herein is used to describe specific embodiments of the invention, but their usage does not delimit the invention, except as outlined in the claims.

To be consistent with the commonly used terminology, whenever possible, the terms used herein will follow the definitions recommended by the Optical Society of America (OCIS codes).

In some embodiments, term “photoacoustic microscopy” refers to a photoacoustic imaging technology that detects pressure waves generated by light absorption in the volume of a material (such as biological tissue) and propagated to the surface of the material. In other words, photoacoustic microscopy is a method for obtaining images of the optical contrast of a material by detecting acoustic or pressure waves traveling from the object. The emphasis is on the micrometer scale image resolution.

In some embodiments, the term “photoacoustic tomography” also refers to a photoacoustic imaging technology that detects acoustic or pressure waves generated by light absorption in the volume of a material (such as biological tissue) and propagated to the surface of the material. The emphasis is sometimes on computer-based image reconstruction although photoacoustic tomography encompasses photoacoustic microscopy.

In some embodiments, the term “piezoelectric detectors” refers to detectors of acoustic waves utilizing the principle of electric charge generation upon a change of volume within crystals subjected to a pressure wave.

In some embodiments, the terms “reflection mode” and “transmission mode” refer to a laser photoacoustic microscopy system that employs the detection of acoustic or pressure waves transmitted from the volume of their generation to the optically irradiated surface and a surface that is opposite to, or substantially different from, the irradiated surface, respectively.

In some embodiments, the term “time-resolved detection” refers to the recording of the time history of a pressure wave with a temporal resolution sufficient to reconstruct the pressure wave profile.

In some embodiments, the term “transducer array” refers to an array of ultrasonic transducers.

In some embodiments, the terms “focused ultrasonic detector,” “focused ultrasonic transducer,” and “focused piezoelectric transducer” refer to a curved ultrasonic transducer with a hemispherical surface or a planar ultrasonic transducer with an acoustic lens attached or an electronically focused ultrasonic array transducer.

In some embodiments, the terms “transducer array” and “phase array transducer” refer to an array of piezoelectric ultrasonic transducers.

In some embodiments, the term “photoacoustic waves” refers to pressure waves produced by light absorption.

In some embodiments, “isosbestic wavelength” refers to a wavelength of light characterized by a hemoglobin absorbance that is essentially equal to an oxyhemoglobin absorbance.

In some embodiments, “non-isosbestic wavelength” refers to a wavelength of light characterized by a hemoglobin absorbance that is not equal to an oxyhemoglobin absorbance.

As will be described below, embodiments of the invention provide a method of characterizing a target within a tissue by focusing one or more laser pulses on the region of interest in the tissue so as to penetrate the tissue and illuminate the region of interest. The pressure waves induced in the object by optical absorption are received using one or more ultrasonic transducers that are focused on the same region of interest. The received acoustic waves are used to image the structure or composition of the object. The one or more laser pulses are focused by an optical assembly, typically including optical fibers, lenses, prisms and/or mirrors, which converges the laser light towards the focal point of the ultrasonic transducer. The focused laser light selectively heats the region of interest, causing the object to expand and produce a pressure wave whose temporal profile reflects the optical absorption and thermo-mechanical properties of the object. In addition to a single-element focused ultrasonic transducer, an annular array of ultrasonic transducers may be used to enhance the depth of field of the imaging system by using synthetic aperture image reconstruction. The assembly of the ultrasonic transducer and laser pulse focusing optics are positioned on a cantilever beam and scanned together, performing fast one- or two-directional sector scanning of the object. The cantilever beam is suspended inside a closed, liquid filled container, which has an acoustically and optically transparent window on a side of the transducer-light delivery optics assembly. The window may be permanent or disposable. The window is positioned on an object surface, where acoustic coupling gel is applied. Neither immersion of the object in water nor movement of the scanner relative to the object surface is necessary to perform imaging. Further, a linear transducer array, focused or unfocused in elevation direction, may be used to accelerate image formation. The signal recording includes digitizing the received acoustic waves and transferring the digitized acoustic waves to a computer for analysis. The image of the object is formed from the recorded acoustic waves.

In addition, embodiments of the invention may also include one or more ultrasonic transducers or a combination thereof. The electronic system includes scanner drivers and controllers, an amplifier, a digitizer, laser wavelength tuning electronics, a computer, a processor, a display, a storage device or a combination thereof. One or more components of the electronic system may be in communication remotely with the other components of the electronic system, the scanning apparatus or both.

The imaging method described herein, which uses a confocal photoacoustic imaging system, is one of the possible embodiments, specifically aimed at medical and biological applications but not limited to these applications. The embodiments of the invention are complementary to pure optical and ultrasonic imaging technologies and may be used for diagnostic, monitoring or research purposes. The main applications of the technology include, but are not limited to, the imaging of arteries, veins, capillaries (the smallest blood vessels), pigmented tumors such as melanomas, hematomas, acute burns, and or sentinel lymphatic nodes in vivo in humans or animals. Embodiments of the invention may use the spectral properties of intrinsic optical contrast to monitor blood oxygenation (oxygen saturation of hemoglobin), blood volume (total hemoglobin concentration), and even the metabolic rate of oxygen consumption; it may also use the spectral properties of a variety of dyes or other contrast agents to obtain additional functional or molecular-specific information. In other words, embodiments of the invention are capable of functional and molecular imaging.

In other aspects, a single-RBC photoacoustic flowoxigraphy (FOG) device is described herein, which can noninvasively image oxygen delivery from single flowing RBCs in vivo with 10 millisecond temporal resolution and 3.4 micrometer spatial resolution. The single-RBC photoacoustic flowoxigraphy (FOG) device uses intrinsic optical absorption contrast from oxy-hemoglobin (HbO₂) and deoxy-hemoglobin (Hb), and therefore, allows label-free imaging. Multiple single-RBC functional parameters, including the total hemoglobin concentration (C_{Hb}), the oxygen saturation (sO₂), the gradient of oxygen saturation (∇sO₂), the flow speed (V_{flow}), and the metabolic rate of oxygen (MRO₂), may be simultaneously quantified in real time. The system works in reflection instead of transmission mode, allowing noninvasive imaging in vivo.

Other embodiments of the invention may be used to monitor possible tissue changes during x-ray radiation therapy, chemotherapy, or other treatment, and may also be used to monitor topical application of cosmetics, skin creams, sun-blocks or other skin treatment products. Embodiments of the invention, when miniaturized, may also be used endoscopically. e.g., for the imaging of atherosclerotic lesions in blood vessels or precancerous and cancerous lesion in the gastrointestinal tract.

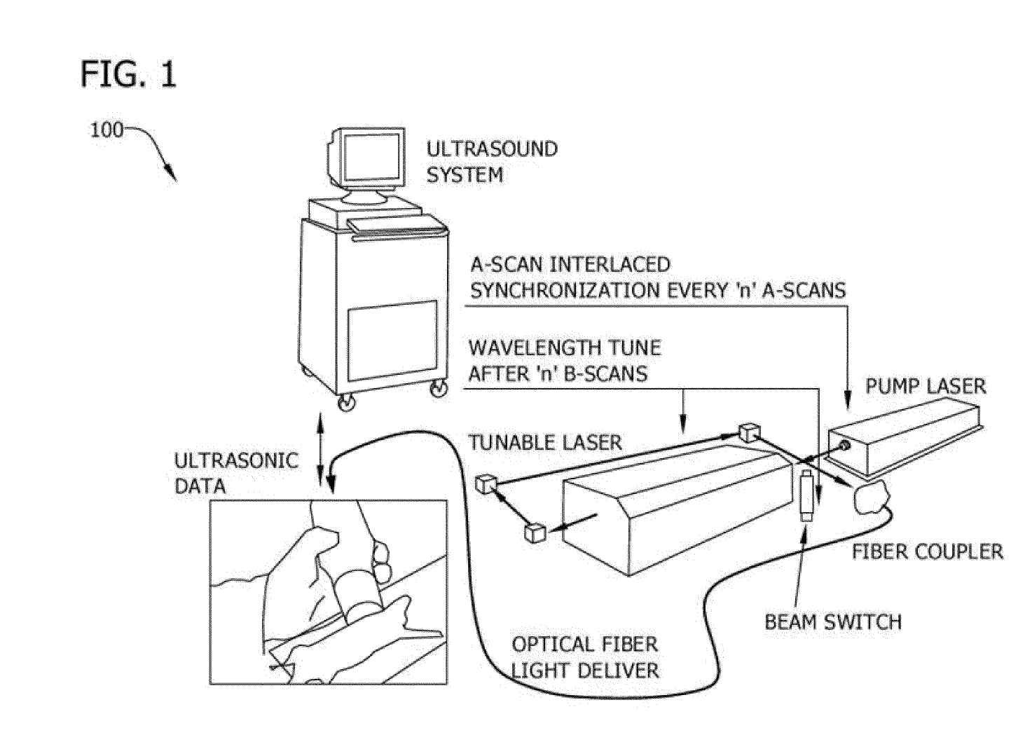

To incorporate photoacoustic imaging into an ultrasonic scanning system or imaging system 100, a photoacoustic excitation source, such as a tunable pulsed dye laser, and a light delivery system are introduced to the ultrasonic scanning system 100 as shown in FIG. 1. The light delivery system, including an optical fiber and light focusing optics, are integrated into the handheld ultrasonic scanner. Light from either the pump laser (before frequency doubling) or the tunable dye laser may be selected with a beam switch and coupled into the optical fiber. The laser must be synchronized with the imaging system 100. In the exemplary embodiment, the imaging system 100 interlaces trigger pulses between the laser and the ultrasonic pulser. The imaging system 100 also controls the emission wavelength of the tunable laser. The light focusing optics is placed inside the ultrasonic scanning head.

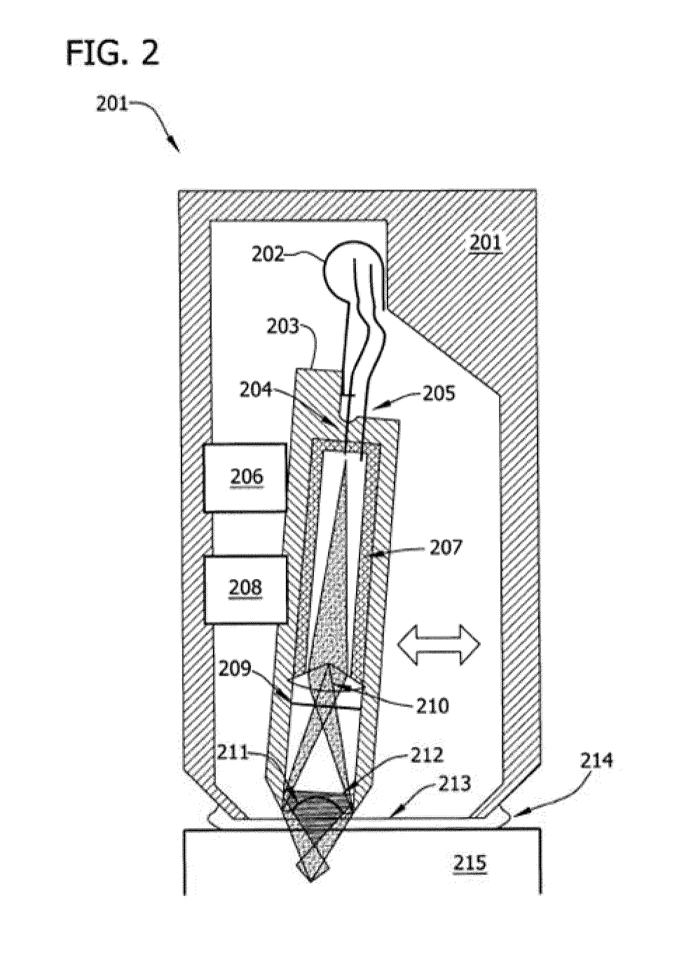

FIG. 2 is a diagram of an exemplary photoacoustic scanner 200 of the imaging system in accordance with one embodiment of the invention. As shown in FIG. 2, scanner 200 is implemented as a handheld device. A dye laser, pumped by a Q-switched pulsed neodymium-doped yttrium lithium fluoride (Nd:YLF) laser delivers approximately 1.0 millijoules (mJ) per pulse to a 0.60-mm diameter optical fiber 204. The laser pulse width is approximately 8.0 nanoseconds (ns), and the pulse repetition rate varies from approximately 0.1 kilohertz (kHz) to approximately 2.0 kHz. The fiber output 204 is coaxially positioned with a focused ultrasonic transducer 211. The concave bowl-shaped transducer 211 has a center frequency of approximately 30.0 megahertz (MHz) and a nominal bandwidth of 100%. The laser light from the fiber 204 is expanded by a conical lens 210 and then focused through an annular hollow cone shaped optical condenser 212, which also serves as a back-plate of the ultrasonic transducer. The optical focal region overlaps with the focal spot of the ultrasonic transducer 211, thus forming a confocal optical dark-field illumination and ultrasonic detection configuration. The photoacoustic setup is mounted inside a hollow cylindrical cantilever beam 203 supported by a flexure bearing 202. The cantilever beam 203 is mounted inside a container 201. The container is filled with immersion liquid and sealed with an optically and acoustically transparent membrane 213. The object 215, e.g., animal or human, is placed outside the container 201 below the membrane 213, and the ultrasonic coupling is further secured by coupling gel 214. The cantilever beam is moved by an actuator 206, and its inclination angle is controlled by a sensor 208. Part of the laser pulse energy is reflected from the focusing optics, such as a conical lens 210, and after multiple reflections from the diffusely reflecting coating of the integrating chamber 207, is detected by a photodetector 205. The signal from the photo-detector 205 is used as a reference signal to take into account energy fluctuations of the laser output. An aperture diaphragm 209 screens the photo-detector 205 from ambient light and sample surface reflection.

Compared to alternative designs, the above design provides the following advantages. First, the high axial stiffness of the cantilever beam increases repeatability of the axial position of the photoacoustic detector. Second, the frictionless flexure bearing pivot decreases the lateral position error of the photoacoustic detector and the mass of the system, thereby decreasing mechanical vibration (noise) of the scanner and increasing its overall mechanical stability. Third, the sealed container design makes the photoacoustic scanner portable and ergonomic, which widens the application field of the photoacoustic technique, especially in medical and biological practice. Fourth, the device performs interlaced acquisition of time-resolved laser-induced pressure waves and reflected ultrasonic pulses, which may be used, for example, to measure the tissue metabolic rate through co-registration of ultrasound pulsed-Doppler and photoacoustic spectral data at high temporal and spatial resolution.

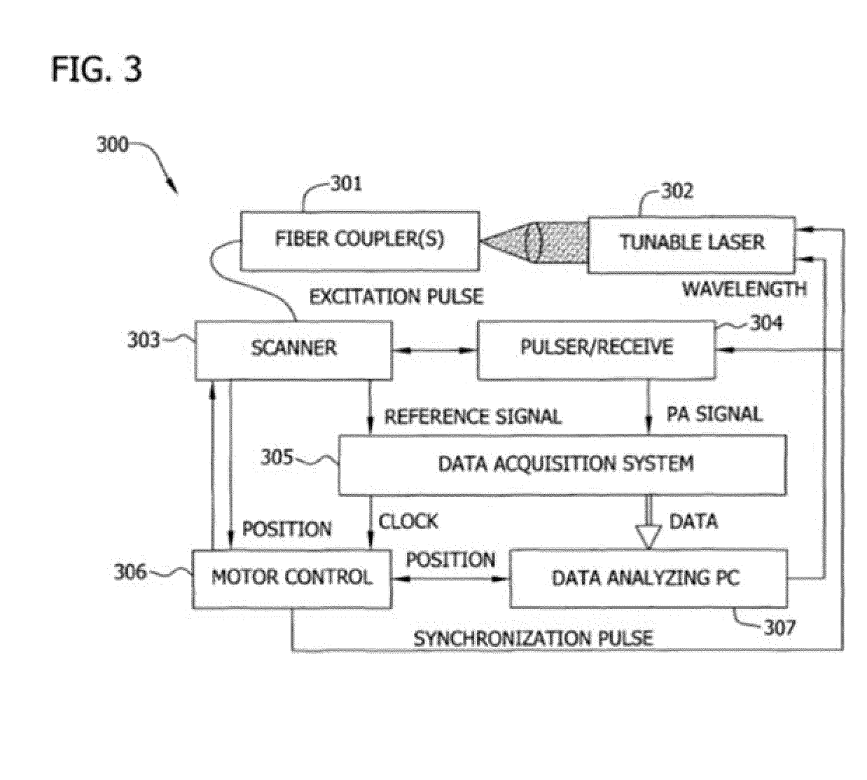

FIG. 3 is a block diagram of an exemplary photoacoustic system 300 that uses dark-field photoacoustic microscopy with sector scanning and quantitative spectroscopic measurement capability in accordance with one embodiment of the invention. The system includes a light delivery subsystem that includes of a tunable pulsed laser subsystem 302, an optical fiber or fibers and the associated fiber coupling optics 301, a scanner 303 that includes a light focusing device and one or more ultrasonic transducers, and an electronic system that may include an ultrasonic pulser/receiver 304, a motion controller 306, a data acquisition system 305, and a data-analyzing computer 307. Depending on the particular application, the photoacoustic system 300 may have an array of peripheral devices (not shown) such as manipulation arm, health and environment monitoring devices, and data storage. The focusing device of the scanner 303 is connected to an output of the fiber coupler 301 via single or multiple optical fibers that receive one or more laser pulses from the tunable laser 302 and focus the one or more laser pulses into a tissue so as to illuminate the tissue. The one or more ultrasonic transducers positioned alongside the focusing optics are focused on the region of interest and receive acoustic or pressure waves induced in the object by the laser light. The electronic system records and processes the received acoustic or pressure waves and controls scanner motion. Ultrasonic transducers may work in two modes, as a receiving transducer for photoacoustic signals and as a pulser/receiver for conventional pulse/echo ultrasonic imaging. The focusing device includes an optical assembly of lenses, prisms, and/or mirrors that expands and subsequently converges the laser light toward the focal point of the one or more ultrasonic transducers.

The dark field confocal photoacoustic sensor is placed on a cantilever beam to perform sector scanning along the tissue surface. The near-simultaneously (e.g., approximately 20.0 microsecond (μs) delayed) recorded photoacoustic and pulse/echo pressure-wave time histories are displayed by the data-analyzing PC 307 versus the photoacoustic sensor position to construct co-registered images of the distribution of the optical and mechanical contrast within the tissue. Depending on the type of scanning (e.g., one or two axis), the device produces cross-sectional (B-scan) or volumetric images of the tissue structure. When the tissue under investigation is an internal organ, the optical fiber and transducer may be incorporated in an endoscope and positioned inside the body.

The data acquisition subsystem 305 produces a clock signal to synchronize all electronic components of the photoacoustic device. The motor controller 306 drives the cantilever beam actuators and measures the current position of the photoacoustic transducer. At transducer locations predefined by the data-analyzing computer 307, the motor controller generates trigger pulses synchronized with the clock signal, which are used to trigger the pulse laser and start the data acquisition sequence.

High-frequency ultrasonic waves generated in the tissue by the laser pulse are recorded and analyzed by the data analyzing computer 307 to reconstruct an image. The shape and dimensions of the optical-contrast tissue structures are generally determined from the temporal profile of the laser-induced ultrasonic waves and the position of the focused ultrasonic transducer. A single axis sector scanning by the ultrasonic transducer positioned within the cantilever beam is used to form a two-dimensional image, and two-axis scanning is used to form a three-dimensional image. However, a transducer array may be used to reduce the time of scanning and light exposure. The following examples are provided for the purpose of illustrating various embodiments of the invention, and are not meant to limit the embodiments of the invention in any fashion.

To obtain functional images, laser pulses from a tunable laser (e.g., a dye laser) are used to illuminate the tissue surface. By switching between several light wavelengths, the optical absorption spectrum of a tissue structure may be measured. This spectrum is influenced by the dispersion of optical absorption and scattering in the object. Nevertheless, in cases where the tissue absorption has definite and distinct spectral features, which is the case, for example, with oxyhemoglobin and deoxyhemoglobin, by using a proper minimization procedure it is possible to separate the contributions of different tissue constituents, and thus permit the measurement of local blood oxygenation in the tissue in order to separate normal and diseased tissues. Similarly, certain tumors may be identified by targeting them with biomolecules conjugated to various contrast agents such as selectively absorbing dyes.

Embodiments of the invention may include any realization of a photoacoustic imaging device which uses a cantilever beam to perform object scanning. The following devices may implement the method described herein: a semi-rigid cantilever beam supported by a flexure bearing, a fixed end flexible cantilever beam, a cantilever beam with two degrees of freedom supported by two perpendicular flexure bearings, and a cantilever beam supported by a flexure bearing attached to a linear scanning stage.

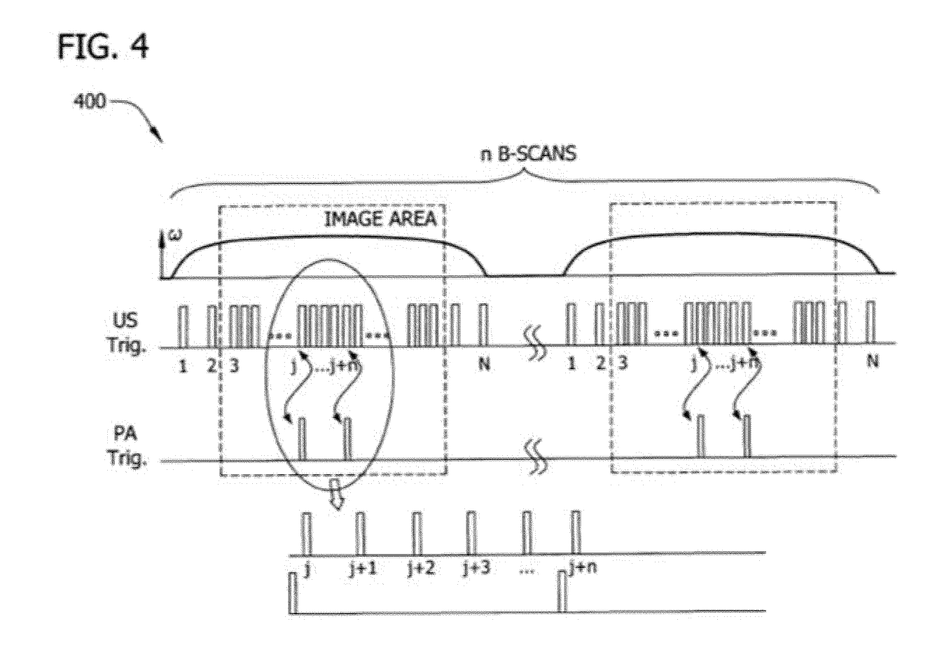

To synchronize the optical and ultrasonic components of the ultrasonic-based photoacoustic imaging system, the ultrasonic system shown in FIGS. 1-3 generates a triggering signal for the pulsed laser as shown in the timing diagram 400 of FIG. 4. The ultrasonic system acquires signals from the ultrasonic transducer and reference photo-detector and superimposes and/or codisplays photoacoustic images and ultrasound pulse-echo images. More specifically, a pump laser produces a pulse energy of approximately 20.0 mJ at a fundamental wavelength of approximately 1056.0 nm, and/or a tunable dye laser produces a pulse energy of greater than approximately 2.0 mJ at a frequency of up to approximately 2.0 kHz. The laser system thus provides approximately 8.0 ns wide laser pulses, which are short enough for the targeted spatial resolution. The ANSI safety limits are satisfied for a pulse energy less than or equal to approximately 2.0 mJ, a diameter of illumination greater than or equal to approximately 6.0 mm, a laser frequency less than or equal to approximately 2.0 kHz, and a scanning step size greater than or equal to approximately 0.1 mm. At 2 kHz PRF, the data acquisition time for a B-scan frame consisting of 200 A-lines is approximately 100.0 ms, yielding a B-scan frame rate of approximately 10.0 Hz. When approximately 20.0 mJ of pulse energy is used for deep penetration, the illumination area is increased to greater than or equal to approximately 1.0 cm2 and the laser PRF decreased to approximately 50.0 Hz. Taking into account the decreased resolution for deep imaging, a B-scan frame rate of approximately 1.0 Hz is achieved if fifty A-lines are acquired to per B-scan.

Moreover, the ultrasonic scanning system generates one photoacoustic imaging synchronization signal for every n pulse-echo ultrasonic triggering pulses (shown as trigger pulses j and j+n in the timing diagram in FIG. 4), where n is approximately the ratio of the ultrasound PRF to the laser PRF. As the ultrasonic scanning progresses into the next frame (Bscan), the laser triggers will be generated in connection with pulse-echo triggers j+1 and j+n+1 correspondingly. After n consecutive frames of scanning, a complete photoacoustic image will be acquired, and the cycle will continue. At this time, the ultrasonic scanning system generates a control word to change the wavelength of the dye laser emission if spectral information is to be collected. Because the photoacoustic imaging system works at a fraction of the frame rate of the ultrasonic system, laser triggers will be simply introduced between consecutive pulse-echo triggers a few microseconds depending on imaging depth (e.g., approximately 20.0 μs for a depth of approximately 30.0 mm) ahead of the corresponding pulse-echo trigger. This lead time will be sufficient for the data acquisition of photoacoustic data before the ultrasonic pulser applies a high voltage to the ultrasonic transducer. This mode of operation does not compromise the pure ultrasonic frame rate while the maximum photoacoustic imaging frame rate is achieved.

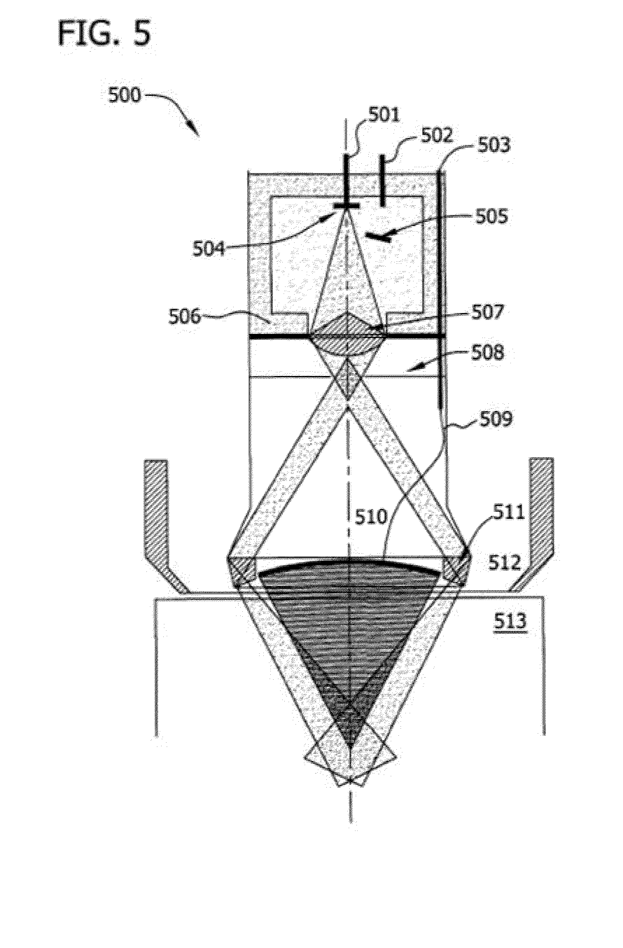

Various examples of photoacoustic scanners will now be described in reference to FIGS. 5-7, wherein the photoacoustic sensor includes an optical focusing device and one or more ultrasonic transducers.

The embodiments of the invention provides fast (e.g., approximately thirty frames per second) high resolution photoacoustic imaging of biological tissues in vivo. This particular embodiment has a lateral resolution as high as approximately 50.0 micrometers (μm) and an imaging depth limit of about 5.0 mm. The image resolution may be further improved by either increasing the frequency and bandwidth of the ultrasonic transducer or increasing the numerical aperture of the optical objective lens. The latter applies when imaging within the depth of one optical transport mean free path is desired. With the help of an ultrasonic array transducer, faster photoacoustic imaging is possible and signal averaging, when needed, is also realistic.

Embodiments of the invention may include any realization of light focusing any kind of mirrors, prisms, lenses, fibers, and diaphragms that may produce illumination directed to the focal area of the focused ultrasonic transducer if sector scanning of the object is performed. Embodiments of the invention may also include any photoacoustic techniques with any light delivery and ultrasonic detection arrangement placed inside a sealed container for scanning, where the container may remain motionless during acquisition of one image frame.

The following devices may be used to implement photoacoustic sensing for the purpose described herein: (1) a bowl-shaper focusing ultrasonic transducer; (2) a flat ultrasonic transducer attached to an acoustic lens; (3) a linear or (4) an annular focused or unfocused ultrasonic transducer array combined with an optical microscope annular condenser which may consist of lenses, mirrors, prisms or their combination. Various examples of the photoacoustic assembly suitable to be placed inside the hollow cantilever beam will now be described in reference to FIGS. 5-7 wherein the focusing assembly includes an optical focusing device and one or more ultrasonic transducers.

A diagram of a photoacoustic sensor assembly 500 of the imaging system in accordance with the main embodiment of the embodiments of the invention is shown in FIG. 5. More specifically, FIG. 5 shows a diagram of one embodiment of a photoacoustic sensor 500 in accordance with the scanner design shown in FIG. 1. A laser pulse is delivered via optical fiber 501, expanded by a conical lens 507, passed around the ultrasonic transducer 510, and focused by a conical prism 511. The transducer 510, focusing optics 507 and 511, optical fibers 501 and 502, and electrical wires connecting the transducer are placed inside the cylindrically shaped cantilever beam 509. In a non-scattering object, the laser energy distribution along the ultrasonic transducer axis would be confined to the transducer's depth of focus. In highly scattering media, the laser energy distribution is broader. The laser light penetrates through the transparent membrane 512 and the surface of the object 513 to a sufficient depth, selectively heating targets in the tissue that have higher optical absorption and producing ultrasonic waves. The ultrasonic waves that propagate toward the tissue surface are detected by an acoustic transducer 510, and digitized and transferred to a computer for data analysis. Part of the energy of the laser pulse is reflected from the lens surface, and the reflected light is homogenized by multiple reflections from the diffusively reflective coating of an integrating chamber 506 and reaches the sensing optical fiber 502. The output of the sensing optical fiber 502 is connected to a photo-detector (not shown). This measurement is used to compensate for the fluctuations in the laser output. An iris diaphragm 508 prevents most ambient light from entering the integrating chamber. An optical absorber 504 absorbs collimated back reflected and ambient light, which enters the integrating chamber through the iris aperture. A baffle 505 shields the sensing fiber from direct exposure to light reflected from the conical lens.

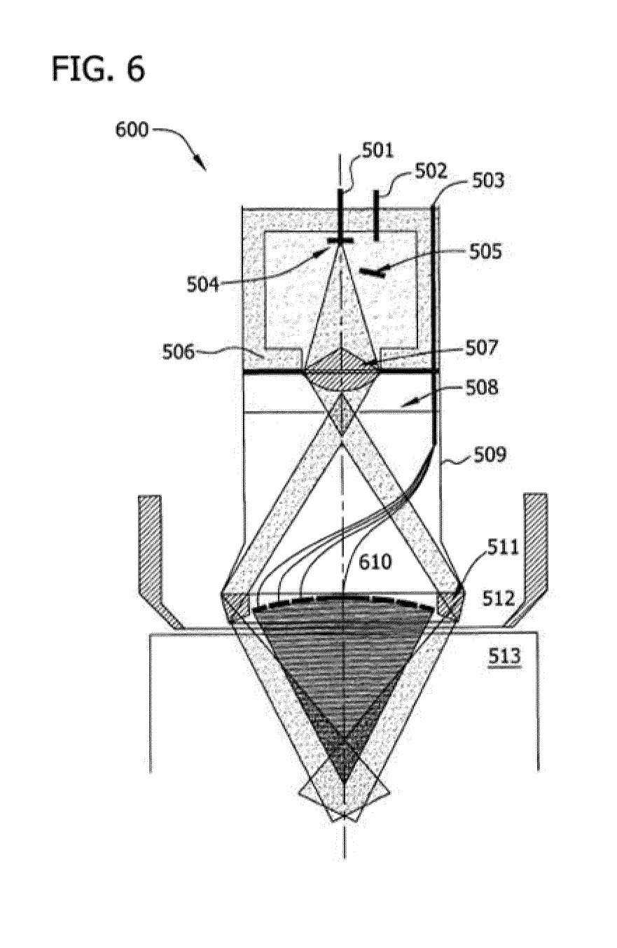

FIG. 6 shows a diagram of another embodiment of a photoacoustic sensor 600 of the imaging system in accordance with FIG. 1. The photoacoustic sensor 600 is similar to photoacoustic sensor 500 (shown in FIG. 5), except that the single-element focused ultrasonic transducer is replaced with a multi-element annular piezoelectric transducer array 610. The ultrasonic transducer array 610 may be dynamically focused to different depths for a single laser pulse by introducing time-offlight-dependent time delays between signals from different transducer elements, thus extending the depth range of the cross-sectional (B-scan) image with high lateral resolution.

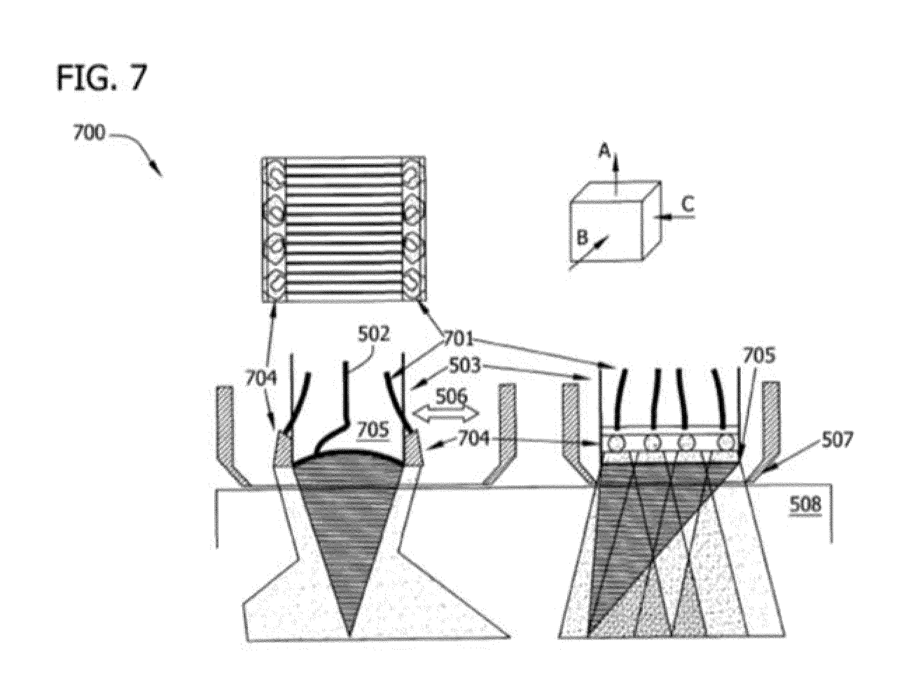

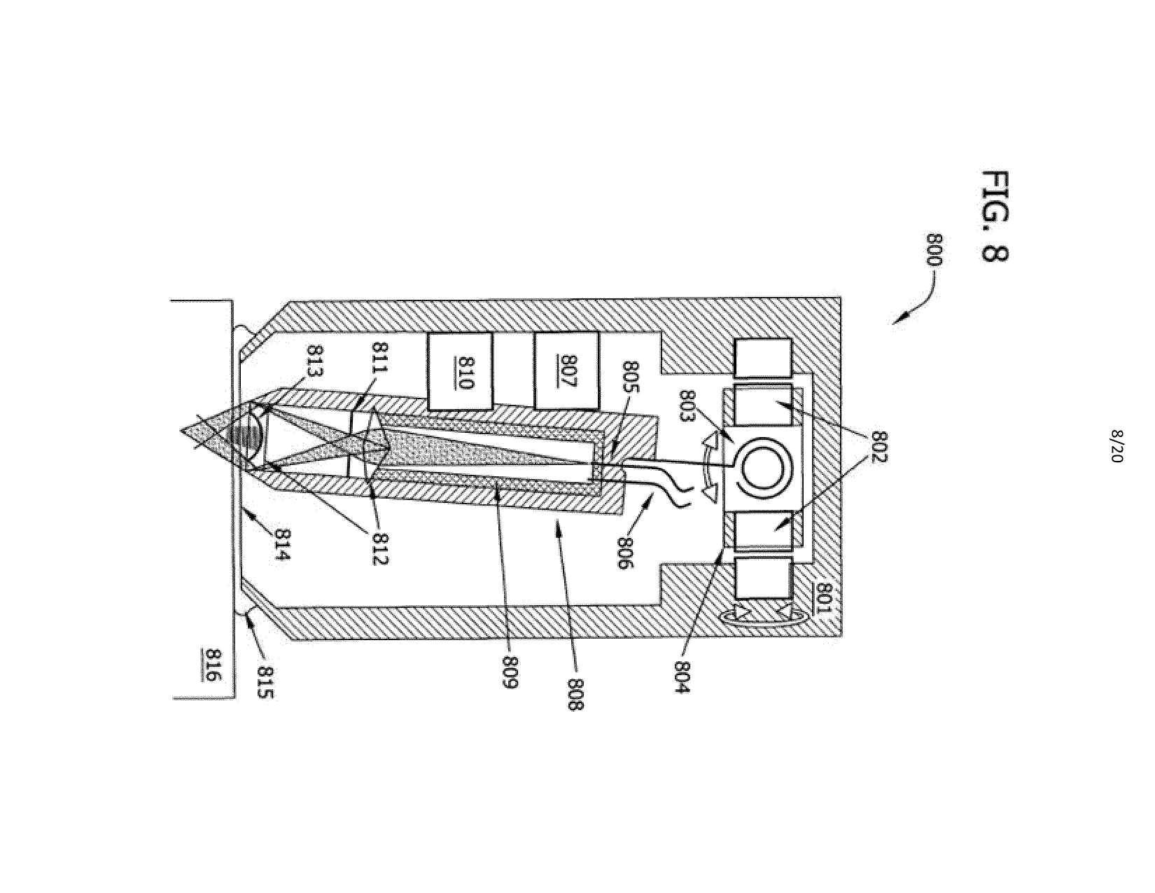

FIG. 7 shows a diagram of yet another embodiment of a photoacoustic sensor 700 of the imaging system 100 shown in FIG. 1. The photoacoustic sensor 700 uses a multitude of optical fibers 701, a system of prisms 704 to deliver light pulses, and a one-dimensional cylindrically focused transducer array 705 to form a photoacoustic B-scan image. In this embodiment, the photoacoustic sensor 700 uses translational symmetry instead of cylindrical symmetry. Unlike the embodiments shown in FIGS. 5 and 6, a wedge-shaped light beam is formed instead of a cone-shaped one, and a linear transducer array 705, similar to one used in medical ultrasonic diagnostics, is used to acquire photoacoustic signals. Using beam forming, such a device may produce a complete photoacoustic B-scan image with a single laser pulse, making possible ultrafast real-time photoacoustic imaging with the B-scan frame rate limited by the pulse repetition rate of the laser. Sector scanning the single row of piezoelectric elements produces volumetric photoacoustic images at potential rates of approximately thirty volumetric frames per second.

FIG. 8 is a block diagram of another embodiment of a photoacoustic scanner 800 that uses sector scanning in two perpendicular directions. A laser pulse is coupled into an optical fiber 805, which is coaxially positioned with the focused ultrasonic transducer 813. With the help of the focusing optics 812, the laser light from the fiber 805 is expanded, passed around the transducer, and then converged towards the ultrasonic focus inside the object under investigation 816. The optical focal region overlaps with the focal spot of the ultrasonic transducer 813, thus forming a confocal optical dark-field illumination and ultrasonic detection configuration. The photoacoustic setup is mounted inside a hollow cylindrical cantilever beam 808 supported by a first flexure bearing 803. The bearing 803 is mounted in a frame 804, which is mounted inside a container 801 on second and third flexure bearings 802. The axis of rotation of the second and third bearings 802 is perpendicular to the axis of rotation of the first bearing 803. Tilting of the cantilever beam 808 in two perpendicular directions results in two dimensional scanning along the object surface. The container 801 is filled with immersion liquid and sealed by an optically and acoustically transparent membrane 814. The object (e.g., animal or human) 816 is placed outside the container 801 below the membrane 814, and ultrasonic coupling is further secured by coupling gel 815. The cantilever beam 808 is moved by an actuator 807, and its inclination angle is controlled by a sensor 810. Part of the laser pulse energy is reflected from the focusing optics, such as conical lens 812, and, after multiple reflections from the diffusely reflective coating of an integrating chamber 809, is transmitted by the sensing optical fiber 806 to a photo-detector (not shown). The signal from the photo-detector is used as a reference signal to offset the energy fluctuations of the laser output. An aperture diaphragm 811 screens the photo-detector from ambient light and sample surface reflection.

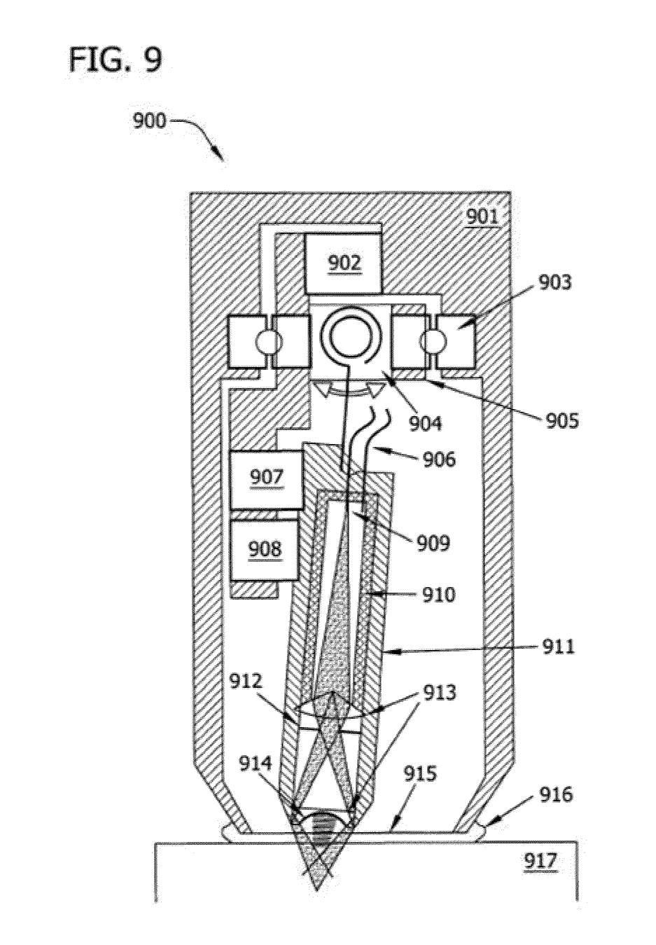

FIG. 9 is a block diagram of another embodiment of a photoacoustic scanner 900 that uses sector scanning in one direction and linear scanning in a perpendicular direction. A laser pulse is coupled into optical fiber 909, which is coaxially positioned with a focused ultrasonic transducer 914. With the help of a focusing optics 913, the laser light from the fiber 909 is expanded, passed around the transducer 914, and then converged towards the ultrasonic focus inside the object under investigation 917. The optical focal region overlaps with the focal spot of the ultrasonic transducer 914, thus forming a confocal optical dark-field illumination and ultrasonic detection configuration. The photoacoustic detector setup is mounted inside a hollow cylindrical cantilever beam 911 supported by a flexure bearing 904, which is mounted in a frame 905. The frame 905 is mounted on a translation stage 903 inside a container 901. The axis of rotation of the bearing 904 is perpendicular to the displacement direction of the translation stage 903. Tilting of the cantilever beam 911 in combination with linear motion in a perpendicular direction results in two dimensional scanning along the object surface. The container 901 is filled with immersion liquid and sealed with optically and acoustically transparent membrane 915. The sample (e.g., animal or human) 917 is placed outside the container 901 below the membrane 915, and the ultrasonic coupling is further secured by coupling gel 916. The cantilever beam 911 is moved by an actuator 907, and its inclination angle is controlled by a sensor 908. The translation stage 903 is moved by a motor 902, which may be a combination of a ball screw, belts, a step motor, a voice coil linear actuator, or piezoelectric actuator. Part of the laser pulse energy is reflected from the focusing optics, such as conical lens 913, and, after multiple reflections from the diffusely reflective coating of an integrating chamber 910, is transmitted by the sensing optical fiber 906 to a photo-detector (not shown). The signal from the photo-detector is used as a reference signal to compensate for the energy fluctuations of the laser output. An aperture diaphragm 912 screens the photo-detector from ambient light and sample surface reflections.

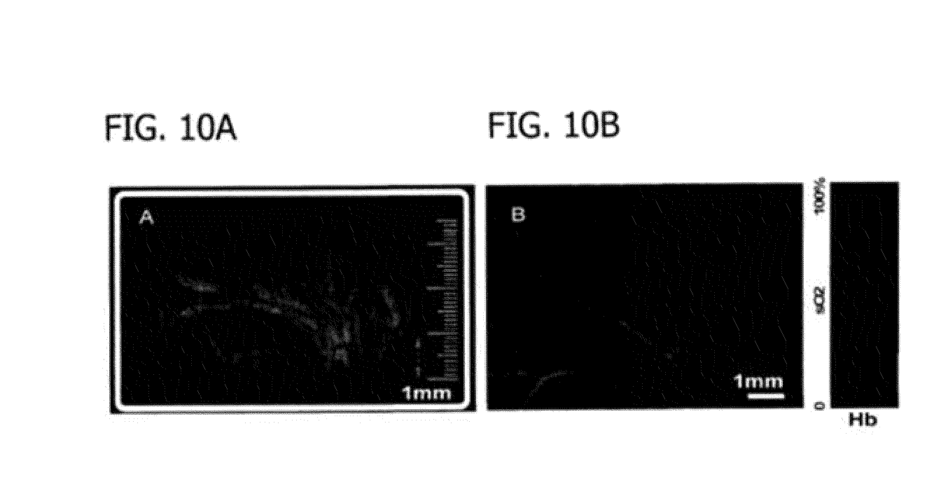

FIG. 10A shows a blood flow image in a mouse prostate taken by an ultrasonic system and FIG. 10B shows a blood oxygenation level image acquired with photoacoustic imaging. More specifically, FIG. 10A shows 3D tumor perfusion and flow architecture in a mouse prostate tumor imaged by an ultrasonic system, and FIG. 10B shows a photoacoustic image of SO₂ in subcutaneous blood vessels in a 200-g Sprague-Dawley rat in vivo. Structural image data reflects the total hemoglobin concentration acquired at 584 nm, color reflects the SO₂. The combination of these two contrasts can shed light on tissue oxygen consumption within the volume of for example a relatively small tumor or small organ, which reflects the metabolic rate of the tissue.

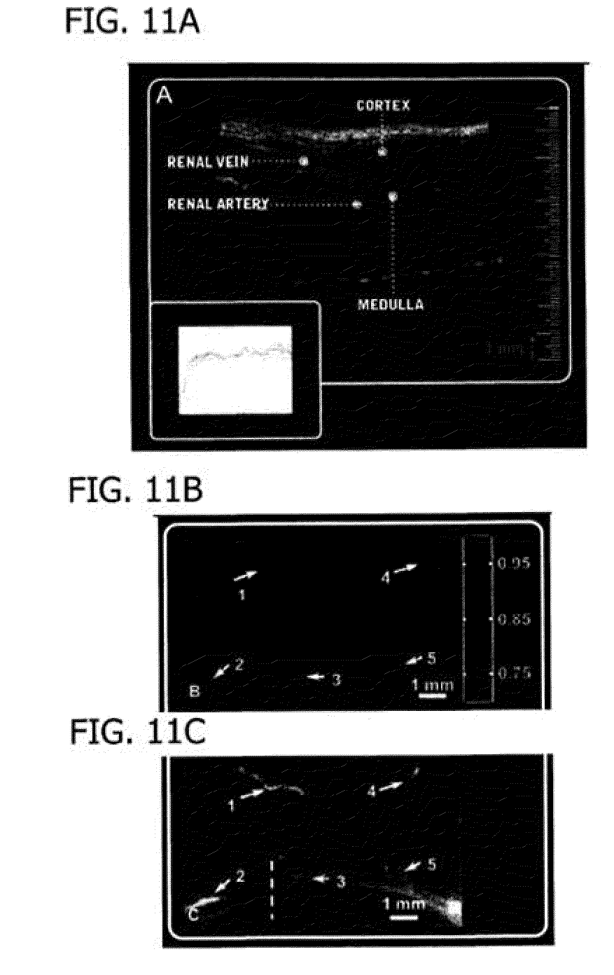

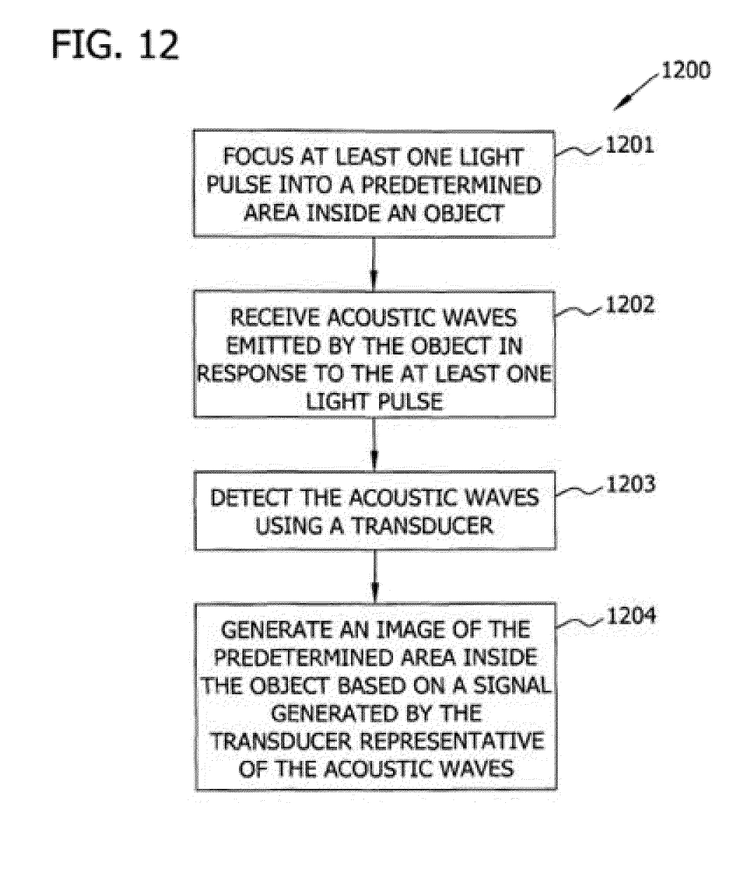

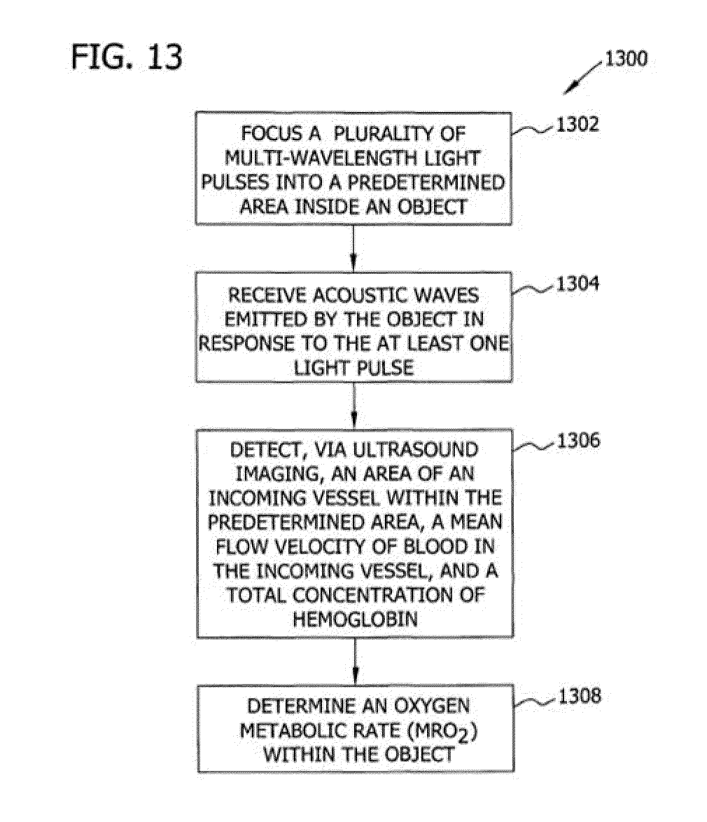

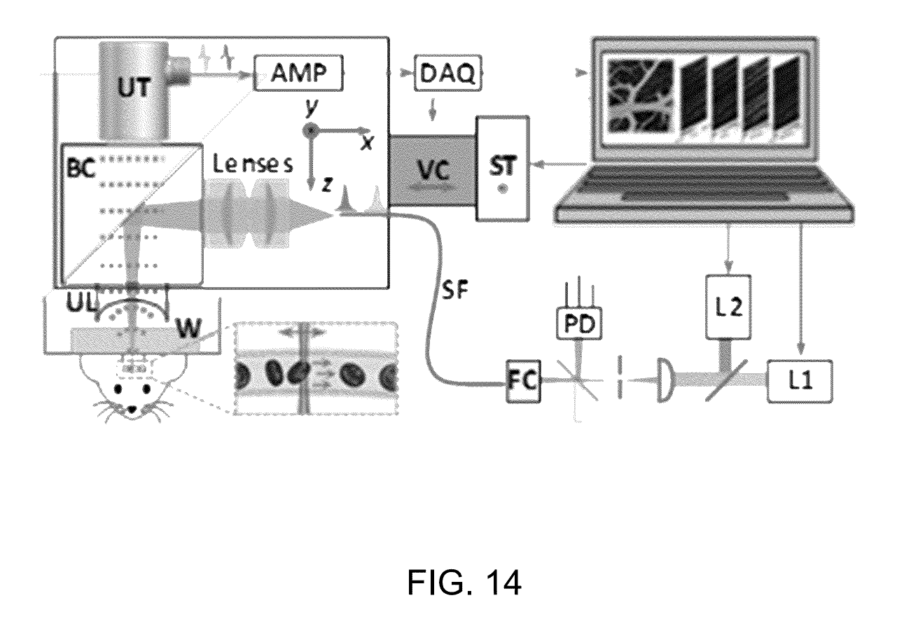

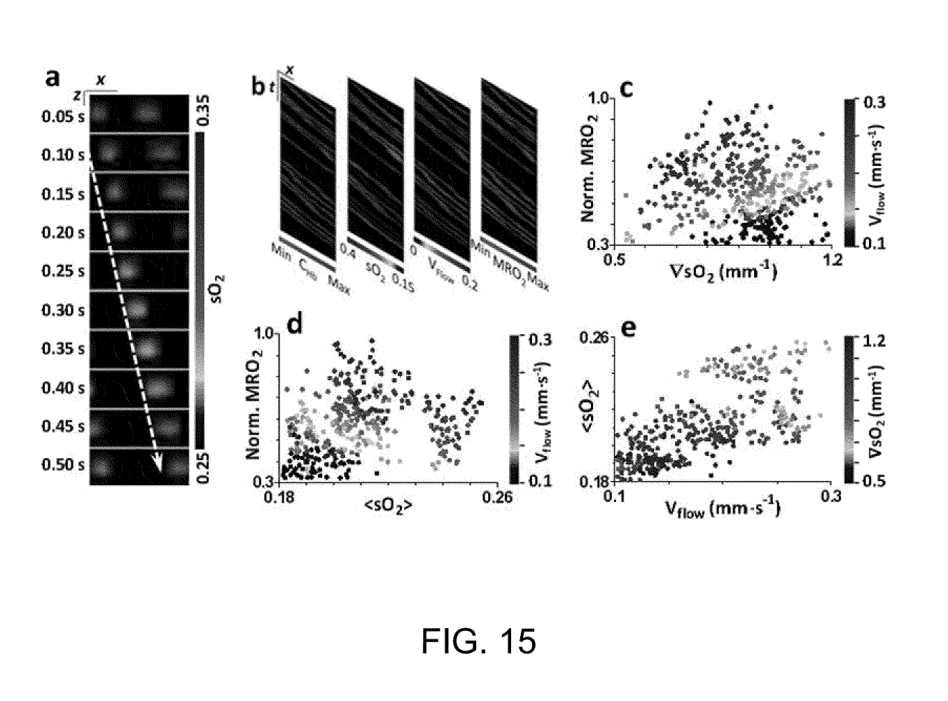

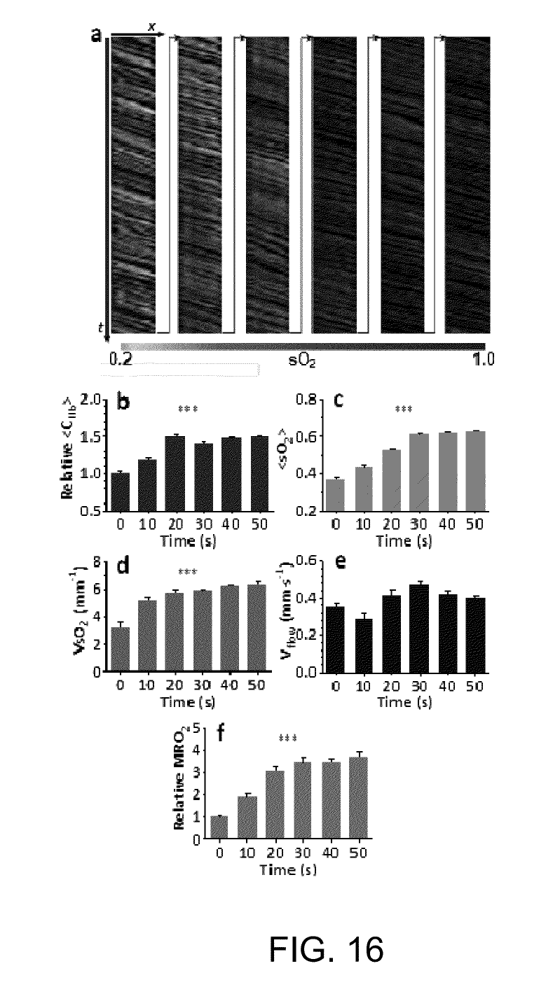

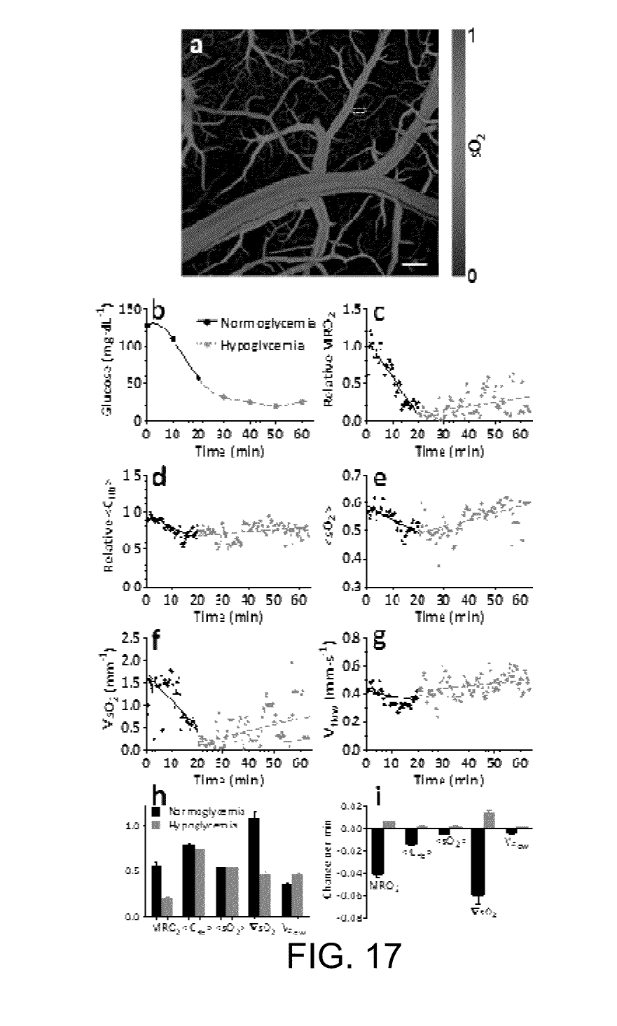

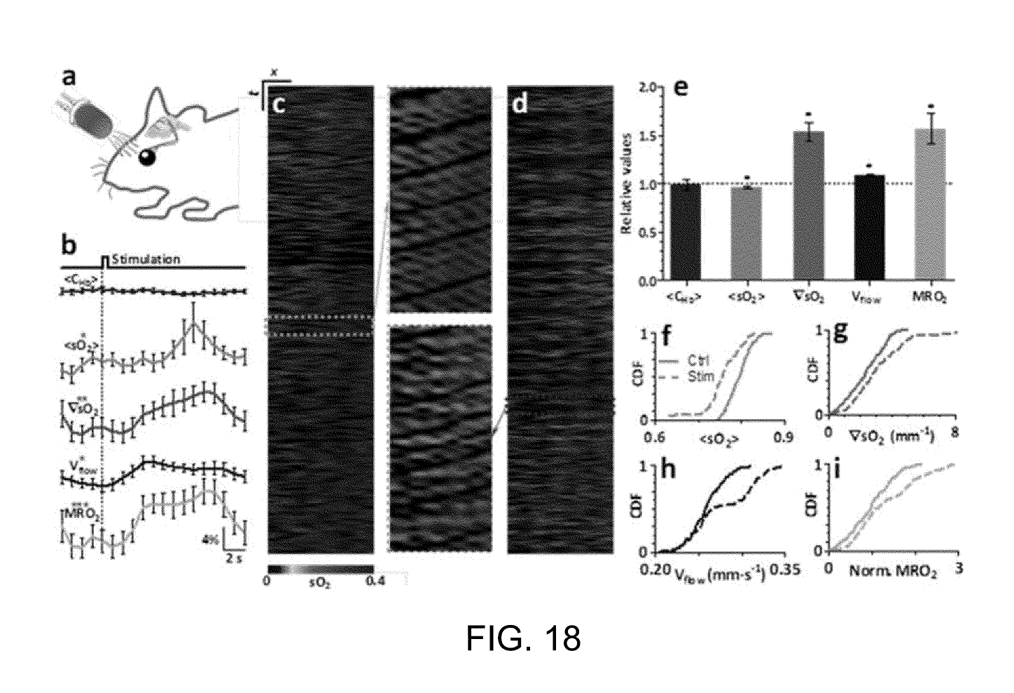

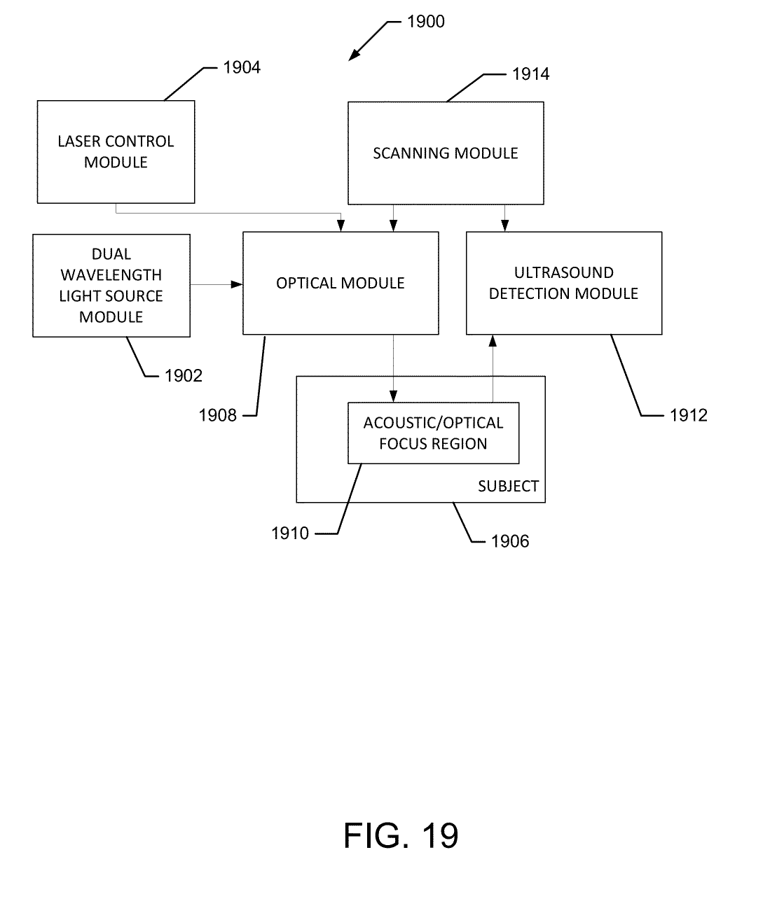

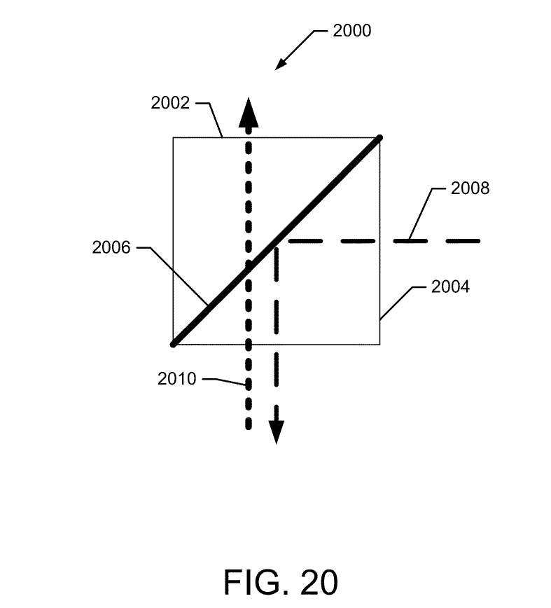

Similarly, a contrast agent enhanced ultrasonic image, as shown in FIG. 11A, taken by an ultrasonic system shows blood perfusion and major veins and arteries but must rely on anatomical cues to distinguish between veins and arteries. By contrast, such a distinction can be made by photoacoustic imaging directly using the imaged oxygen saturation of hemoglobin, as shown in FIG. 11B. This distinction is confirmed as shown in FIG. 11C, which shows ex-vivo microsphere-perfusion image of arterioles (red) and venules (blue).

By recording photoacoustic signals obtained at various optical wavelengths, the optical absorption spectrum of the object may be measured. The optical absorption coefficient is dominated by the absorption of hemoglobin in many cases. Because two forms of hemoglobin—oxygenated and deoxygenated—have distinctly different absorption spectra, one may recover the partial concentrations of the two forms of hemoglobin. This value may be used to quantify the oxygen saturation of hemoglobin and the relative total concentration of hemoglobin. Of course, this example merely illustrates the principle, which may be extended to the measurement of other optical absorbers using two or more excitation optical wavelengths.

Because of the fast frame rate, the device in the embodiments of the invention may combine blood flow measurement into and out of regions of interest using the pulse-Doppler technique with blood oxygenation measurements to estimate oxygen metabolism in tissues and organs. The oxygen metabolic rate (MRO₂) is the amount of oxygen consumed in a given tissue region per unit time per 100 g of tissue or of the organ of interest. In typical physiological conditions, since hemoglobin is the dominant carrier of oxygen, the key measure of blood oxygenation is the oxygen saturation of hemoglobin (SO₂). Therefore, we have

MRO₂∞(SO_{2,in}—SO_{2,out})·C_{Hb}·Aᵢₙ· v̅ᵢₙ Eq. (1)

here, Aᵢₙ, is the area of the incoming vessel, v̅ᵢₙ, is the mean flow velocity of blood in the incoming vessel, and C_{Hb} is the total concentration of hemoglobin. While Aᵢₙ, and v̅ᵢₙ, may be estimated using ultrasound imaging, SO₂, and relative C_{Hb}, may be estimated from multi-wavelength photoacoustic methods.

FIG. 12 is a flowchart 1200 illustrating an exemplary photoacoustic tomography imaging method that characterizes a tissue by focusing 1201 one or more laser pulses on a region of interest in the tissue and illuminating the region of interest. More specifically, the laser pulses are emitted from collimating optics mounted on a cantilever beam that is flexibly mounted within a handheld device. In one embodiment, the cantilever beam is a semi-rigid cantilever beam supported by a flexure bearing. In another embodiment, the cantilever beam is a fixed-end flexible cantilever beam. In another embodiment, the cantilever beam is mounted with two degrees of freedom and is supported by perpendicular flexure bearings. In yet another embodiment, the cantilever beam is supported by a flexure bearing that is coupled to a linear scanning cage. Acoustic waves induced in the object by optical absorption are received 1202 and a signal is generated 1203 representative of the acoustic waves using one or more ultrasonic transducers that are focused on the same region of interest. The signal is then used to image 1204 the structure or composition of the object. The one or more laser pulses are focused by an optical assembly, typically including lenses, prisms, and/or mirrors, which converges the laser light towards the focal point of the ultrasonic transducer. The focused laser light selectively heats the region of interest, causing the object to expand and produce a pressure wave having a temporal profile that reflects the optical absorption and thermo-mechanical properties of the object. In addition to a single-element, focused ultrasonic transducer, an annular array of ultrasonic transducers may be used to enhance the depth of field of the imaging system by using synthetic aperture image reconstruction. The assembly of the ultrasonic transducer and laser pulse focusing optics are positioned on a cantilever beam and scanned together, performing fast one-directional or two-directional sector scanning of the object. The cantilever beam is suspended inside a closed, liquid-filled container, which has an acoustically and optically transparent window on a side of the transducer-light delivery optics assembly. The window is positioned on an object surface and acoustic coupling gel is applied. The received acoustic waves are digitized and the digitized acoustic waves are transmitted to a computer for analysis. An image of the object is then formed from the digitized acoustic waves.

FIG. 13 is a flowchart 1300 illustrating an exemplary method for determining an oxygen metabolic rate (MRO₂) within a biological tissue using a handheld device. A plurality of multi-wavelength light pulses are focused 1302 on a region of interest in the tissue and illuminating the region of interest. More specifically, the laser pulses are emitted from collimating optics mounted on a cantilever beam that is flexibly mounted within a handheld device. In one embodiment, the cantilever beam is a semi-rigid cantilever beam supported by a flexure bearing. In another embodiment, the cantilever beam is a fixed-end flexible cantilever beam. In another embodiment, the cantilever beam is mounted with two degrees of freedom and is supported by perpendicular flexure bearings. In yet another embodiment, the cantilever beam is supported by a flexure bearing that is coupled to a linear scanning cage. Acoustic waves induced in the object by optical absorption are received 1304 using one or more ultrasonic transducers that are focused on the same region of interest. The signal is then used to detect 1306 an area of an incoming vessel within the predetermined area, a mean flow velocity of blood in the incoming vessel, and a total concentration of hemoglobin. The area of the incoming vessel and the mean flow velocity are based on measurements obtained by ultrasound imaging, and the total concentration of hemoglobin is based on measurements obtained by the plurality of multi-wavelength light pulses. The MRO₂ is determined 1308 based on the area of the incoming vessel, the mean flow velocity of blood in the incoming vessel, and the total concentration of hemoglobin using Equation (1) as explained above. The MRO₂ is the amount of oxygen consumed in a given tissue region per unit time per 100 g of tissue or of the organ of interest. In typical physiological conditions, since hemoglobin is the dominant carrier of oxygen, the key measure of blood oxygenation is the oxygen saturation of hemoglobin (SO₂). The one or more laser pulses are focused by an optical assembly, typically including lenses, prisms, and/or mirrors, which converges the laser light towards the focal point of the ultrasonic transducer. The focused laser light selectively heats the region of interest, causing the object to expand and produce a pressure wave having a temporal profile that reflects the optical absorption and thermo-mechanical properties of the object. In addition to a single-element, focused ultrasonic transducer, an annular array of ultrasonic transducers may be used to enhance the depth of field of the imaging system by using synthetic aperture image reconstruction. The assembly of the ultrasonic transducer and laser pulse focusing optics are positioned on a cantilever beam and scanned together, performing fast one-directional or two-directional sector scanning of the object. The cantilever beam is suspended inside a closed, liquid-filled container, which has an acoustically and optically transparent window on a side of the transducer-light delivery optics assembly. The window is positioned on an object surface and acoustic coupling gel is applied. The received acoustic waves are digitized and the digitized acoustic waves are transmitted to a computer for analysis. An image of the object is then formed from the digitized acoustic waves.

By implementing photoacoustic imaging capabilities on a commercial ultrasound system, ultrasound and photoacoustic pulse sequences may be interleaved to obtain (1) structural images from ultrasound B-mode scans, (2) functional images of total hemoglobin concentration from photoacoustic scans, (3) functional images of hemoglobin oxygen saturation (SO₂) from photoacoustic scans, and (4) images of melanin concentration from photoacoustic scans as well. Therefore, photoacoustic imaging will significantly enrich the contrast of ultrasound imaging and provide a wealth of functional information.

A single-RBC photoacoustic flowoxigraphy (FOG) device is provided in another aspect. In this aspect, the device delivers laser pulses of two different wavelengths separated by a pulse separation period of about 20 μs. This separation period is sufficiently brief to enable pulses of two different wavelengths to illuminate the same single moving RBC. The acoustic signals elicited by the single RBC in response to the laser pulses of two different wavelengths may be analyzed using pulse oximetry methods similar to those described herein above to simultaneously determine a variety of functional parameters including, but not limited to: total hemoglobin concentration (C_{Hb}), oxygen saturation (sO₂), gradient of oxygen saturation (∇sO₂), flow speed (V_{flow}), and metabolic rate of oxygen (MRO₂), and any combination thereof.

Single-RBC FOG may be an effective tool for in vivo imaging of the oxygen exchange between single RBCs and their local environments. The optical diffraction-limited lateral spatial resolution and the >100-Hz two-dimensional imaging rate enable resolution of single flowing RBCs in real time. The short, 20 μs, dual wavelength switching time, enables the detection of oxygenation in flowing RBCs. Other time intervals may be used. During fast scanning, this imaging modality maintains the confocal alignment between the optical and acoustic foci. This provides superior SNR compared with pure optical scanning, and may be of great importance for sensitive functional imaging. The single-RBC FOG also has the advantage of label-free imaging, relying on intrinsic optical absorption contrast from HbO₂ and Hb. This feature avoids the use of contrast agents that might be chemically toxic, phototoxic, radioactive, or disruptive to the imaging targets. Taking full advantage of the single-RBC FOG, the dynamic processes of single RBCs delivering oxygen to local cells and tissues in vivo at multiple anatomical sites, including the brain, may be directly imaged.

The single-RBC FOG is able to simultaneously measure multiple functional parameters, which include C_{Hb}, sO₂, ∇sO₂, V_{flow}, and MRO₂. Such a capability can uncover the relationships between these tightly related parameters, and provide a comprehensive view of cell and tissue oxygenation with high spatiotemporal resolution. Dynamics of single-RBC oxygen release may be imaged under normoxia and during a transition from hypoxia to hyperoxia. Experimental results show that the RBC oxygen delivery may be regulated by V_{flow} and sO₂.

Single RBCs, as basic oxygen carriers, play a key role in oxygenating most cells and tissues. To date, the lack of technologies available for direct functional imaging of single RBCs in vivo has been a major limiting factor in studies of oxygen metabolism at high temporal and spatial resolution. The single-RBC FOG demonstrated here has broken through this limitation by directly imaging the oxygen release processes from single RBCs, as well as allowing for simultaneous measurement of C_{Hb}, sO₂, ∇sO₂, V_{flow}, and MRO₂. This advance in single-RBC FOG may open new avenues for studying fundamental principles in oxygen metabolism and related diseases. This device may be used in clinical or pre-clinical applications, to diagnose or study some microvascular diseases, such as septic shock, sickle cell anemia, and circulating tumor cells; or some metabolic diseases such as diabetes and cancer.

FIG. 19 is a schematic illustration showing the arrangement of the components and devices of the single-RBC photoacoustic flowoxigraphy (FOG) device 1900 in one aspect. The device 1900 may include a dual wavelength light source module to produce a series of isosbestic laser pulses at an isosbestic wavelength, an isosbestic pulse width of less than about 10 ns and an isosbestic pulse repetition rate of at least 2 kHz and a series of non-isosbestic laser pulses at a non-isosbestic wavelength, a non-isosbestic pulse width of less than about 10 ns and a non-isosbestic pulse repetition rate of at least 2 kHz. In an aspect, the isosbestic wavelength may be any wavelength with a hemoglobin absorbance that is essentially equal to an oxyhemoglobin absorbance. Non-limiting examples of suitable isosbestic wavelengths include: 532 nm, 548 nm, 568 nm, 587 nm, and 805 nm. In this same aspect, the non-isosbestic wavelength may be any wavelength with a hemoglobin absorbance that is not equal to the oxyhemoglobin absorbance. In another aspect, the isosbestic wavelength is about 532 nm and the non-isosbestic wavelength is about 560 nm.

In another aspect, the dual wavelength light source module 1902 may include an isosbestic laser (not shown) to produce the series of isosbestic laser pulses and a non-isosbestic laser (not shown) to produce the series of non-isosbestic laser pulses. Any known laser device with the capable of producing laser pulses at the wavelengths, pulse widths, and pulse repetition rates as described herein above may be used. Various suitable laser devices are described herein previously.

Referring again to FIG. 19, the device 1900 may further include a laser control module 1904 to trigger the delivery of each isosbestic laser pulse and each non-isosbestic laser pulse, wherein each isosbestic laser pulse is delivered at a pulse separation period of about 20 μs before or after each adjacent non-isosbestic laser pulse. The 20 μs pulse separation period is sufficiently brief so that both an isosbestic laser pulse and a non-isosbestic laser pulse may illuminate each individual RBC as it moves through a vessel in the subject 1906. In addition, the 20 μs pulse separation period provides sufficient time for each acoustic signal corresponding to each pulse to be emitted and detected without interfering with previous or subsequent acoustic signals induced by previous or subsequent laser pulses. Methods of controlling and timing the operation of the lasers is provided previously herein.

Referring again to FIG. 19, the device 1900 may further include an optical module 1908 to direct the series of isosbestic laser pulses and the series of non-isosbestic laser pulses through an optical focus region 1910 in a cylindrical beam with a beam cross-sectional diameter of less than about 10 μm. The optical focus region, as described previously herein, may typically include a capillary or other vessel or tissue of interest within the subject 1906. In one aspect, the optical module 1908 may include an optical fiber (not shown) connected to the isosbestic laser and the non-isosbestic laser at a first end. The optical fiber may receive both isosbestic and non-isosbestic laser pulses in a combined stream and direct the combined pulse streams to the optical focus region 1910. Any known optical fiber may be included in the optical module 1908 including, but not limited to a single-mode fiber. The optical fiber may further include optical couplers to direct the output of the lasers into the optical fiber.

In another aspect, the optical module 1908 may further include additional optical components (not shown) to focus the laser pulses delivered by the optical fiber into a beam with a beam diameter of less than about 10 μm through the optical focus region 1910. Any known optical components described previously herein may be incorporated into the optical module 1908 including, but not limited to lenses, mirrors, prisms, condensers, and any other suitable known optical component. In one aspect, the optical module 1908 may further include a pair of optical lenses including, but not limited to a pair of achromatic doublets with a numerical aperture in water of about 0.1. In this aspect, the additional optical components maybe operatively attached to the optical fiber at a second end of the optical fiber opposite to the first end of the optical fiber.

Referring again to FIG. 19, the device 1900 may further include an ultrasound detection module 1912 to detect acoustic signals generated within the optical focus region 1910 in response to the series of isosbestic and non-isosbestic laser pulses. The ultrasound detection module may include any ultrasound detector (not shown) capable of detecting an acoustic signal within an acoustic focus region 1910 that is aligned with the optical focus region 1910. Any suitable known ultrasound transducer may be incorporated into the ultrasound detection module including any of the ultrasound transducers described herein previously. In one aspect, the ultrasound detection module 1910 may include a focused ultrasound transducer with a central frequency of about 50 MHz. In this aspect, this focused ultrasound transducer may result in an axial spatial resolution of about 15 μm.

In order to maintain the acoustic and optical focus regions 1910 in an aligned orientation, the optical module 1908 may further include an additional element to reflect the focused laser pulses in a direction that is essentially aligned with the detection axis of the focused ultrasound transducer. In one aspect, the optical module 1908 may further include an acoustically transparent optical reflector to transmit acoustic signals from the acoustic focus region 1910 to the focused ultrasound transducer and to reflect the series of isosbestic and non-isosbestic laser pulses from the optical assembly to the optical focus region 1910.

FIG. 20 is a schematic diagram of an acoustically transparent optical reflector focusing assembly 2000 in one aspect. The assembly 2000 may include a first right-angle prism 2002 and a second first right-angle prism 2004 with a sub-micron reflective aluminum coating layer 2006 sandwiched between the two prisms 2002/2004, forming a reflective plane. The aluminum layer 2006 reflects incoming laser pulses 2008 in a downward direction, but also transmits acoustic signals 2010 propagating upward from the optical/acoustic focus region. As a result, the directions of the incoming laser pulses 2008 and the outgoing acoustic signals 2010 are aligned, resulting in a reduced signal-to-noise ratio (SNR).

Referring again to FIG. 19, the device 1900 may further include a scanning module 1914 to move the optical module 1908 and the ultrasound detection module 1912 in a linear scanning pattern. In this arrangement, the device 1900 may obtain imaging data over a linear transect that may be used to obtain a two-dimensional plane image corresponding to a vertical slice extending the length of the linear scan and the depth corresponding to the focus range of the ultrasound transducer of the ultrasound detection module 1912. Because both the optical module 1908 and the ultrasound detection module 1912 are translated together in a synchronized manner, the alignment of the laser pulses and the acoustic signals is maintained, resulting in higher quality imaging data as discussed herein previously. In order to obtain images in real time, the scanning module may have a scanning rate of at least 100 linear scans per second. In one aspect, the scanning module 1912 may include any linear scanner described herein previously capable of rapid scanning. In another aspect, the scanning module 1912 may include a voice-coil scanner with a scanning rate of at least 100 linear scans per second.

FIG. 14 is a schematic diagram illustrating the arrangement of elements and components of a single-RBC photoacoustic flowoxigraphy (FOG) device in one aspect. In this aspect, two lasers L1 and L2 are employed to periodically generate two 20-μs-apart laser pulses at 560 nm (non-isosbestic wavelength) and 532 nm (isosbestic wavelength), respectively with pulse widths of less than 10 ns. Both lasers L1 and L2 operate at a 2-kHz pulse repetition rate. Other wavelengths may also be used as long as the deoxy-hemoglobin and oxy-hemoglobin have different absorption coefficients.

The two laser beams are merged into a single-mode optical fiber SF by way of a fiber coupler FC and then delivered to a PA probe. The energy of each laser pulse is detected by a biased photodiode PD for pulse-to-pulse calibration. The laser beam from the fiber SF is focused onto targets through a pair of optical lenses (numerical aperture in water: 0.1), an acoustic-optical beam combiner BC, and an ultrasound lens UL. The optical lenses can be adjusted to accurately align the acoustic and optical foci. The acoustic-optical beam combiner BC, which is composed of two prisms and a coated aluminum layer in the middle, reflects light, but transmits sound. The tight optical focus provides a 3.4-μm lateral spatial resolution. Laser-excited PA signals are collected by the ultrasound lens UL, transmitted through the acoustic-optical beam combiner BC, and detected by a high-frequency ultrasound transducer UT.

By way of non-limiting example, the ultrasound transducer may be a model V214 Olympus NDT, 50 MHz central frequency transducer which provides an axial spatial resolution of 15 μm. The PA signals are amplified by an amplifier AMP, filtered and digitized at 500 MHz by a digitizer DAQ.

Referring again to FIG. 14, the PA probe is mounted onto a fast voice-coil linear scanner VC to enable acquisitions of at least 100 cross-sectional (B-scan) images per second. Mechanically scanning the entire PA probe maintains the acoustic-optical confocal alignment, and therefore achieves higher signal-to-noise ratio (SNR) than pure optical scanning in a fixed acoustic focus. A field-programmable gate array card (PCI-7830R, National Instrument, not shown) may be programmed to synchronize the trigger signals and motion control commands in an aspect. By fast scanning the PA probe along a segment of a capillary, single RBCs flowing through the field of view may be imaged, and the amount of oxygen bound to each RBC may be directly measured.

At each position, the two laser pulses sequentially excite nearly the same region of the target to acquire two depth-resolved PA signals (A-lines). Taking 10-mm·s⁻¹ flow speed as an example, the target may move 0.2 μm during the wavelength switching, which is a small distance relative to the spatial resolution of the device. As a result, image artifacts due to movement of the individual RBCs during wavelength switching are minimal. Because HbO₂ and Hb have molar extinction coefficients of different spectral characteristics, and because PA signals are linearly related with the concentrations of HbO₂ and Hb at low excitation laser energy (<100 nJ per pulse), the relative concentrations of HbO₂ and Hb may be computed from the PA signals of the same RBC excited at 532 nm and 560 nm pulses. The relative CHb and sO₂ of single RBCs may readily be calculated from the HbO₂ and Hb concentrations using methods similar to those described herein previously. To quantify the local oxygen delivery, the average hemoglobin concentration <C_{Hb}> may be computed by averaging C_{Hb} over the imaged segment of a capillary of the subject.

In an aspect, when sO₂ reaches a dynamic equilibrium, the amount of oxygen delivered by RBCs may be assumed to be equal to the oxygen consumed by the perfused tissues; hence, the MRO₂ can be determined from Eqn. (2):

MRO₂=k× [symbol] C_{Hb} [symbol] ×∇sO₂×V_{flow}, (2)

where k is a constant coefficient related to the hemoglobin oxygen binding capacity and the weight of the local tissue surrounding a unit length of the capillary. Note that, the ∇sO₂, V_{flow}, and MRO₂ represent the averages of the capillary segment within the field of view.

### EXAMPLES

The following examples illustrate various aspects of the disclosure.

### Example 1

### Quantitative Measurement of Oxygen Release from Single Red Blood Cells In Vivo

To demonstrate the measurement of oxygen release from individual RBCs in vivo, the following experiment was conducted. Using a device similar to the single-RBC photoacoustic flowoxigraphy (FOG) device described in FIG. 14, a set of B-scan images were acquired at varied time points, shown in FIG. 15A were obtained to record real-time oxygen delivery as single RBCs flowed from the left to the right side of the field of view. The oxygen release from single RBCs was clearly imaged cell by cell. Taking advantage of the ultra-short wavelength switching time, fast scanning speed, and high spatial resolution, C_{Hb}, sO₂, ∇ₛ∘₂, V_{flow}, and MRO₂ can simultaneously be quantified from images of single RBCs, as shown in FIG. 15B. By operating a 532-nm single-wavelength laser at a 20-kHz pulse repetition rate, three-dimensional imaging of flowing single RBCs with a 20-Hz rate may be achieved. Other rates may be used.

The ears of nude mice (Hsd:Athymic Nude-FoxINU, Harlan Co., 4-6 weeks old, 20-25 g of weight) and the brains of white mice (Hsd:ND4 Swiss Webster, Harlan Co., four to six weeks old, 20-25 g of weight) were imaged for all in vivo studies. During imaging, mice were placed on top of a 37° C. heating pad, secured with a head holder, and anaesthetized with isoflurane (Isothesia, Isoflurane USP, Buttler Animal Health Supply). Ultrasound gel was applied between the imaging areas and the water tank. After the ear imaging experiments, the mice naturally recovered. Brain imaging experiments were studied through a small craniotomy. After the brain imaging experiments, the mice were sacrificed via cervical dislocation under deep anesthesia. The laser pulse energy used for the brain imaging was 40-50 nJ usually, and up to 100 nJ when deep RBCs were imaged. To offset skin attenuation, 80 nJ was used when the mouse ear was imaged. Note that the current experimental setup can image capillary segments within a small angle (<10°) from the voice-coil scanning axis x-axis). For capillaries outside the angular range, either the scanner or the animal was rotated to reduce the angle.

### Example 2

### Oxygen Delivery Regulated by V_{flow} and sO₂ Under Normoxia

In order to study the mechanisms that regulate oxygen delivery, single RBCs in mouse brain capillaries were imaged at a 20-Hz B-scan rate while the mice were breathing air mixed with isoflurane using a device similar to the device used in Ex. 1. Even under normoxia, oxygen delivery fluctuates within a range. The imaged capillaries were 60-150 μm deep from the top surface of the brain cortex, and had segments of 30-60 μm in length within the B-scan window. More than 6000 B-scan images at each wavelength were acquired. Multiple functional parameters from the single RBC images were simultaneously calculated and averaged every 20 B-scans.

FIGS. 15C-15E summarize the relationships among <sO₂>, ∇sO₂, V_{flow}, and MRO₂. In FIG. 15C, it was observed that MRO₂ increases with both ∇sO₂ and V_{flow} as expected from Eq. (1). While ∇sO₂ is related to the amount of oxygen released by each RBC, V_{flow} determines the rate of RBCs flowing through the capillary segment. FIG. 15D shows that increasing MRO₂ increases V_{flow} if <sO₂> is maintained constant, but decreases <sO₂> if V_{flow} is held constant. From FIGS. 15C and 15D, it was also observed that, for constant MRO₂, increasing V_{flow} decreases ∇sO₂ and increases <sO₂>. FIG. 15E shows that a decrease of either V_{flow} or <sO₂> is correlated with increasing ∇sO₂.

From these observations, two mechanisms that regulate oxygen release from single RBCs to local tissue under normoxia can be identified. The first one is via sO₂ while V_{flow} and C_{Hb} are held constant. When the local tissue consumes more oxygen (i.e., increases the MRO₂), ∇sO₂ in the capillary increases; consequently, <sO₂> decreases. The other mechanism is via V_{flow} while sO₂ and C_{Hb} are held constant. When the tissue demands more oxygen, it is known that V_{flow} is actively increased so that more RBCs flow through the capillary within a given time period. Because increasing V_{flow} shortens the time for oxygen to diffuse from blood plasma to local tissue, the increasing trend of ∇sO₂ due to the first mechanism is moderated, which allows each RBC to carry oxygen further downstream along the capillary.

### Example 3

### Dynamic Imaging of Oxygen Delivery Under a Transition from Hypoxia to Hyperoxia

Using the device of Ex. 1, the dynamic oxygen delivery process in the mouse ear was imaged under a transition from systemic hypoxia to hyperoxia. Initially, the mouse was breathing in hypoxic gas (5% O₂) for over 10 minutes. When the animal reached a stable systemic hypoxic state, the hypoxic gas was altered to pure oxygen, and immediately started at time 0 to acquire B-scan images at 20 Hz along a segment of a capillary. As shown in FIG. 16A, a dramatic increase in single RBC sO₂ was observed within 60 seconds. Single-RBC functional parameters, including <C_{Hb}>, <sO₂>, ∇sO₂, V_{flow}, and MRO₂, were plotted in FIGS. 16B-16F. Each parameter was computed from the images of single RBCs and averaged over every second. Every 10 data points (10 seconds) were grouped for comparison. Statistical tests show that <C_{Hb}>, <sO₂>, ∇sO₂, and MRO₂ increased by 49%±3%, 71%±2%, 96%±7%, and 270%±22%, respectively, but V_{flow} did not change significantly.

In contrast to the correlation in normoxia, the correlation between the <sO₂> and ∇sO₂ during the transition from hypoxia to hyperoxia is positive. When the inspired gas was switched from low to high oxygen concentration, the capillary sO₂ rapidly increased, and the capillary pO₂ increased along with the sO₂. However, the pO₂ in the surrounding tissue changed more slowly than that in blood. As the enhanced radial gradient in pO₂ increased oxygen diffusion from capillary blood to tissue, the ∇sO₂ was steepened.

### Example 4

### Glucose-Associated Oxygen Metabolism in the Brain

The device of Ex. 1 was used to study glucose-associated oxygen metabolism at high resolution. As shown in FIG. 17A, the sO₂ was imaged over a large field of view in the mouse brain to identify a capillary of interest. Insulin (30 Ul per kilogram) was subcutaneously injected into the mouse to induce a systemic decrease in blood glucose. Immediately after the injection, single-RBC functional images were acquired at 100 Hz for over 60 minutes. The blood glucose concentration was measured with a glucose meter (Freestyle Lite, Abbott Diabetes Care Inc.) by drawing blood once every 10 minutes from the mouse tail. FIG. 4b shows the decrease of the blood glucose level with time. FIGS. 17C-17G summarize the multiple functional parameters computed from the single-RBC images and averaged over every second. It was observed that the blood glucose level determined the local MRO₂. The results, especially the MRO₂, show striking biphasic responses.

The MRO₂ data in FIG. 17C was fitted with a bi-segmental linear regression model according to Eqn (3):

$$y = \{ \begin{matrix}{k}_{1} t + {c}_{1} & if t \leq {t}_{0} \\ {k}_{2} ( t - {t}_{0} ) + {y}_{0} & if t > {t}_{0}\end{matrix} \qquad Eqn ( 3 )$$

where y₀=k₁t₀+c₁, and t₀ is the separation time point between the two linear segments. At the fitted t₀ equal to 19.6 minutes, the blood glucose concentration is 58.5 mg/dL, which agrees well with the dividing point between normoglycemia and hypoglycemia determined independently. Therefore, the quantitative measurements of glucose concentration, MRO₂, <C_{Hb}>, <sO₂>, ∇sO₂, and V_{flow} were divided into two phases accordingly as illustrated in FIGS. 17B-17G. The first phase was defined as normoglycemia (glucose concentration ≧58.5 mg/dL), and the second phase was defined as the hypoglycemia (glucose concentration ≦58.5 mg/dL). The data of each phase were averaged and fitted to a linear regression model to compare the means and slopes as summarized in FIGS. 17H and 17I.

In the normoglycemia phase, MRO₂ decreased with the decreasing glucose concentration (FIG. 17C). The other functional parameters including <C_{Hb}>, <sO₂>, ∇sO₂, and V_{flow} decreased as well. Upon transition from normoglycemia to hypoglycemia, the flow speed was actively increased to compensate for the extremely low glucose metabolism (FIG. 17G). Consequently, MRO₂ and the associated functional parameters started to climb.

### Example 5

### Imaging of Neuron—Single-RBC Coupling

Study of neurovascular coupling has gained broad interest because hemodynamics can be used as an important surrogate to explore neuroscience and study brain disorders. However, existing imaging modalities are limited by either poor spatial resolution or the inability to directly measure MRO₂. Here, the device of Ex. 1 was applied to study coupling between visual neural activity and single RBC functions in the brain.

As shown in FIG. 18A, optical stimulation from a bright white LED was applied to the left eye of a white mouse. Target capillaries were imaged in the right visual cortex region of the brain through a craniotomy. FIG. 18B are graphs summarizing transient responses of single RBCs to single visual stimulations (0.5 second flashing). Each functional parameter is first computed from images of single RBCs, and then averaged over one second for 104 trials. Statistical analyses showed that <sO₂>, ∇sO₂, V_{flow}, and MRO₂ changed significantly after visual stimulation, but <C_{Hb}> did not show obvious changes. An increase in flow speed was observed after stimulation, reaching its peak 4-10 seconds after initial stimulation. The single-RBC <sO₂> exhibited a biphasic response: it remained unchanged within 3-5 seconds after stimulation, then proceeded to rise and peaked after 8-10 seconds. The response of ∇sO₂ and MRO₂ also showed similar behaviors as <sO₂>. This result revealed a clear process of functional hyperemia in the target capillary evoked by visual stimulation.

The coupling between neurons and single RBCs was also imaged under continuous visual stimulation by flashing the LED light at 1 Hz. Capillary segments in the visual cortex region were imaged at 20-100 Hz, varied according to the blood flow speed. FIGS. 18C and 18D show representative sO₂ images acquired without and with continuous visual stimulation, respectively. It was found that the <sO₂> decreased after 3 minutes of continuous visual stimulation. The MRO₂ was also measured while alternating the continuous visual stimulus on and off. Although the individual data points varied considerably, the mean MRO₂ value in each on or off period correlated well with the applied stimulations. All of the single RBC functional parameters, including <C_{Hb}>, <sO₂>, ∇sO₂, V_{flow}, and MRO₂, were compared between without and with continuous visual stimulations. Each of the parameters was normalized to its mean value computed from the control images (without stimulation) and plotted as relative values in FIG. 18E. While the <C_{Hb}> did not have significant changes, the <sO₂> decreased by 4%±0.8%, and the ∇sO₂, V_{flow}, and MRO₂ increased by 53%±6%, 8%±1%, and 56%±9%, respectively.

With the single-RBC resolution, the probability distributions of the single-RBC functional parameters were further quantified. FIGS. 18F-18I show the cumulative distribution functions (CDFs) of the significantly changed single-RBC functional parameters. With continuous stimulation, it was observed that the single-RBC functions exhibited different distributions from those in the control experiment, i.e., lower <sO₂>, higher ∇sO₂, V_{flow}, and MRO₂, which are consistent with the data shown in FIG. 18E.

It will be understood that the particular embodiments described herein are shown by way of illustration and not as limitations of the invention. The principal features of this invention may be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All of the compositions and/or methods disclosed and claimed herein may be made and executed without undue experimentation in light of the present disclosure. While the compositions and methods of this invention have been described in terms of embodiments, it will be apparent to those of skill in the art that variations may be applied to the compositions and/or methods and in the operations or in the sequence of operations of the method described herein without departing from the concept, spirit and scope of the invention. All such similar substitutes and modifications apparent to those skilled in the art are deemed to be within the spirit, scope and concept of the invention as defined by the appended claims.

It will be understood by those of skill in the art that information and signals may be represented using any of a variety of different technologies and techniques (e.g., data, instructions, commands, information, signals, bits, symbols, and chips may be represented by voltages. currents, electromagnetic waves, magnetic fields or particles, optical fields or particles, or any combination thereof). Likewise, the various illustrative logical blocks, modules, circuits, and algorithm operations described herein may be implemented as electronic hardware, computer software, or combinations of both, depending on the application and functionality. Moreover, the various logical blocks, modules, and circuits described herein may be implemented or performed with a general purpose processor (e.g., microprocessor, conventional processor, controller, microcontroller, state machine or combination of computing devices), a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. Similarly, operations of a method or process described herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. Although embodiments of the invention have been described in detail, it will he understood by those skilled in the art that various modifications may be made therein without departing from the spirit and scope of the invention as set forth in the appended claims.

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

What is claimed is:

1. A device for real-time spectral imaging of single moving red blood cells in a subject in vivo, the device comprising:
an isosbestic laser to deliver a series of isosbestic laser pulses at an isosbestic wavelength, an isosbestic pulse width of less than about 10 ns and an isosbestic pulse repetition rate of at least 2 kHz;
a non-isosbestic laser to deliver a series of non-isosbestic laser pulses at a non-isosbestic wavelength, a non-isosbestic pulse width of less than about 10 ns and a non-isosbestic pulse repetition rate of at least 2 kHz;
an optical fiber to direct the series of isosbestic laser pulses and the series of non-isosbestic laser pulses to an optical assembly;
the optical assembly to focus the series of isosbestic laser pulses and the series of series of non-isosbestic laser pulses into a beam with a beam cross-sectional diameter of less than about 10 μm through an optical focus region; and
a laser controller to trigger the delivery of each isosbestic laser pulse and each non-isosbestic laser pulse, wherein each isosbestic laser pulse is delivered at a pulse separation period of about 20 μs before or after each adjacent non-isosbestic laser pulse.

2. The device of claim 1, wherein:
the isosbestic wavelength is a wavelength with a hemoglobin absorbance that is essentially equal to an oxyhemoglobin absorbance;
the isosbestic wavelength is chosen from 532 nm, 548 nm, 568 nm, 587 nm, and 805 nm; and
the non-isosbestic wavelength is any wavelength with the hemoglobin absorbance that is not equal to the oxyhemoglobin absorbance.

3. The device of claim 2, wherein the isosbestic wavelength is about 532 nm and the non-isosbestic wavelength is about 560 nm.

4. The device of claim 1, wherein the optical assembly comprises a pair of optical lenses comprising two achromatic doublets with a numerical aperture in water of about 0.1.

5. The device of claim 1, further comprising a focused ultrasound transducer with an acoustic focus region that is aligned with the optical focus region and a central frequency of at least 10 MHz.

6. The device of claim 5, wherein the central frequency is about 50 MHz and the focused ultrasound transducer further comprises an axial spatial resolution of about 15 μm.

7. The device of claim 5, further comprising a linear scanner to move the optical assembly and the focused ultrasound transducer in a linear scanning pattern.

8. The device of claim 7, wherein the linear scanner is a voice-coil scanner with a scanning rate of at least 100 linear scans per second.

9. The device of claim 5, further comprising an acoustically transparent optical reflector to transmit acoustic signals from the acoustic focus region to the focused ultrasound transducer and to reflect the series of isosbestic and non-isosbestic laser pulses from the optical assembly to the optical focus region.

10. The device of claim 9, wherein the acoustically transparent optical reflector comprises a first prism and a second prism, wherein a first face of the first prism and a second face of the second prism are arranged on opposite sides of an aluminum layer forming a planar optical reflector aligned at an angle of 45° relative to an axis of the optical assembly.

11. A system for real-time spectral imaging of single moving red blood cells in a subject in vivo, the system comprising:
a dual wavelength light source module to produce a series of isosbestic laser pulses at an isosbestic wavelength, an isosbestic pulse width of less than about 10 ns and an isosbestic pulse repetition rate of at least 2 kHz and a series of non-isosbestic laser pulses at a non-isosbestic wavelength, a non-isosbestic pulse width of less than about 10 ns and a non-isosbestic pulse repetition rate of at least 2 kHz;
an optical module to direct the series of isosbestic laser pulses and the series of non-isosbestic laser pulses through an optical focus region in a cylindrical beam with a beam cross-sectional diameter of less than about 10 μm; and
a laser control module to trigger the delivery of each isosbestic laser pulse and each non-isosbestic laser pulse, wherein each isosbestic laser pulse is delivered at a pulse separation period of about 20 μs before or after each adjacent non-isosbestic laser pulse.

12. The system of claim 11, wherein the dual wavelength light source module comprises an isosbestic laser to produce the series of isosbestic laser pulses and a non-isosbestic laser to produce the series of non-isosbestic laser pulses.

13. The system of claim 11, wherein:
the isosbestic wavelength is a wavelength with a hemoglobin absorbance that is essentially equal to an oxyhemoglobin absorbance;
the isosbestic wavelength is chosen from 532 nm, 548 nm, 568 nm, 587 nm, and 805 nm; and
the non-isosbestic wavelength is any wavelength with the hemoglobin absorbance that is not equal to the oxyhemoglobin absorbance.

14. The system of claim 13, wherein the isosbestic wavelength is about 532 nm and the non-isosbestic wavelength is about 560 nm.

15. The system of claim 11, wherein the optical module comprises an optical fiber operatively connected to the isosbestic laser and the non-isosbestic laser at a first end and operatively connected to a pair of optical lenses comprising two achromatic doublets with a numerical aperture in water of about 0.1 at a second end opposite to the first end of the optical fiber.

16. The system of claim 15, further comprising an ultrasound detection module to detect acoustic signals generated within the optical focus region in response to the series of isosbestic and non-isosbestic laser pulses, wherein the ultrasound detection module comprises a focused ultrasound transducer with a central frequency of about 50 MHz and an ultrasound focus region that is aligned with the optical focus region.

17. The system of claim 16, wherein the optical module further comprises an acoustically transparent optical reflector to transmit acoustic signals from the acoustic focus region to the focused ultrasound transducer and to reflect the series of isosbestic and non-isosbestic laser pulses from the optical assembly to the optical focus region.

18. The system of claim 17, further comprising a scanning module to move the optical module and the ultrasound detection module in a linear scanning pattern, wherein the scanning module comprises a voice-coil scanner with a scanning rate of at least 100 linear scans per second.

19. The system of claim 17, wherein the system obtains images of the single moving red blood cells at an axial spatial resolution of about 15 μm and a lateral spatial resolution of about 3.4 μm.

20. The system of claim 17, wherein the system simultaneously obtains one or more functional parameters of the single moving red blood cells using a pulse oximetry method, wherein the one or more functional parameters are chosen from: total hemoglobin concentration, oxygen saturation, gradient of oxygen saturation, flow speed, metabolic rate of oxygen, and any combination thereof.
